SEQUENCE LISTING (23 sequences)

SEQ ID NO: 1 - DNA, 25 bp, Artificial Sequence
  A synthetic primer
gttcacaaccatctaaagcaagatg

SEQ ID NO: 2 - DNA, 19 bp, Artificial Sequence
  A synthetic primer
gttgcatggctggtgcagg

SEQ ID NO: 3 - DNA, 15 bp, Artificial Sequence
  A synthetic primer
cgtgcaagcgcccag

SEQ ID NO: 4 - DNA, 25 bp, Artificial Sequence
  A synthetic primer
ctccaccaacttactgtttcattct

SEQ ID NO: 5 - DNA, 20 bp, Artificial Sequence
  A synthetic primer
gtaacccgttgaaccccatt

SEQ ID NO: 6 - DNA, 20 bp, Artificial Sequence
  A synthetic primer
ccatccaatcggtagtagcg

SEQ ID NO: 7 - DNA, 21 bp, Artificial Sequence
  A synthetic primer
gctgcccaataccaggtcaac

SEQ ID NO: 8 - DNA, 21 bp, Artificial Sequence
  A synthetic primer
tggtgggagaaatgctgtatg

SEQ ID NO: 9 - DNA, 15 bp, Artificial Sequence
  A synthetic primer
cgtgcaagcgcccag

SEQ ID NO: 10 - DNA, 25 bp, Artificial Sequence
  A synthetic primer
ctccaccaacttactgtttcattct

SEQ ID NO: 11 - DNA, 20 bp, Artificial Sequence
  A synthetic primer
gtaacccgttgaaccccatt

SEQ ID NO: 12 - DNA, 20 bp, Artificial Sequence
  A synthetic primer
ccatccaatcggtagtagcg

SEQ ID NO: 13 - DNA, 15 bp, Artificial Sequence
  A synthetic primer
cgtgcaagcgcccag

SEQ ID NO: 14 - DNA, 25 bp, Artificial Sequence
  A synthetic primer
ctccaccaacttactgtttcatcct

SEQ ID NO: 15 - DNA, 20 bp, Artificial Sequence
  A synthetic primer
gtaacccgttgaaccccatt

SEQ ID NO: 16 - DNA, 20 bp, Artificial Sequence
  A synthetic primer
ccatccaatcggtagtagcg

SEQ ID NO: 17 - RNA, 46 nt, Artificial Sequence
  A synthetic RNA sequence
gggagaggguuuaaucuguacgaaaguacugauuggauccgcaagg

SEQ ID NO: 18 - RNA, 46 nt, Artificial Sequence; identical to SEQ ID NO: 17
  A synthetic RNA sequence

SEQ ID NO: 19 - RNA, 55 nt, Artificial Sequence
  A synthetic RNA sequence
gggagaggguuuaauuaaaagucgacgaaagucgucgcuaauuggauccgcaagg

SEQ ID NO: 20 - RNA, 21 nt, Artificial Sequence
  A synthetic RNA sequence
ugcugcugcugcugcugcugc

SEQ ID NO: 21 - RNA, 20 nt, Artificial Sequence
  A synthetic RNA sequence
ggccggccgaaaggccggcc

SEQ ID NO: 22 - RNA, 49 nt, Artificial Sequence
  A synthetic RNA sequence
cccagacuaaccugucucgcuuuuccccuccgcugcggccacuccuuaa

SEQ ID NO: 23 - RNA, 36 nt, Artificial Sequence
  A synthetic RNA sequence
cugcugcugcugcugcugcugcugcugcugcugcug

(19) United States
(12) Patent Application Publication
(10) Pub. No.: US 2018/0296532 A1
(43) Pub. Date: Oct. 18, 2018

(54) RNA-Focused Small Molecules for Inhibiting RNA Toxicity in Myotonic Dystrophy

(71) Applicant: The Scripps Research Institute, La Jolla, CA (US)

(72) Inventors: Matthew D. Disney, Jupiter, FL (US); Suzanne Rzuczek, Jupiter, FL (US)

(21) Appl. No.: 15/760,526

(22) PCT Filed: Sep. 16, 2016
(86) PCT No.: PCT/US16/52259

§ 371 Date: Mar. 15, 2018

Related U.S. Application Data
(60) Provisional application No. 62/220,747, filed on Sep. 18, 2015.

(51) Int. Cl. A61K 31/4184; A61K 31/444; A61P 21/00

(52) U.S. Cl. CPC A61K 31/4184; A61P 21/00; A61K 31/444

(57) ABSTRACT

The invention provides compounds and their pharmaceutical compositions according to Formula (I) that are useful for inhibiting RNA toxicity, such as in the treatment of myotonic dystrophy type 1, wherein W¹, W², W³, W⁴, L¹, L², and Cy are defined herein.

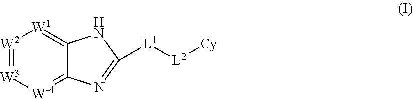

### CROSS-REFERENCE TO RELATED APPLICATIONS

This application claims the benefit of priority to U.S. Provisional Patent Application No. 62/220,747, which was filed on Sep. 18, 2015, the entire contents of which are incorporated by reference as if fully set forth herein.

### BACKGROUND

RNA's diverse and essential biological functions have cemented it as an important class of targets for therapeutics and chemical probes. For example, long, non-coding RNAs, microRNAs, riboswitches, and antisense transcripts function to regulate gene expression.⁽¹⁻⁶⁾ Oligonucleotides, which can be designed by simple Watson-Crick base pairing rules, are commonly employed to target RNA. Indeed, antisense and RNAi-based oligonucleotides have been used successfully to drug malfunctioning RNAs in both cells and animals, showing that RNAs are indeed viable therapeutic targets.⁽⁷⁻¹⁰⁾ Unfortunately, oligonucleotides are generally not cell permeable or drug-like, and efficient, general delivery systems have been elusive.

As an alternative to oligonucleotide-based probes, various approaches have been developed to identify small molecules that target RNA. We have designed small molecules to bind an RNA of interest by examining its secondary structural motifs and comparing them to a database of known and annotated RNA motif-small molecule interactions.⁽¹¹⁾ Structure-based approaches include mimicking natural substrates for riboswitches,⁽¹²⁻¹⁴⁾ designing small molecules to interact with hydrogen bond donors and acceptors in RNA grooves,⁽¹⁵⁻¹⁷⁾ and mimicking interactions between RNAs and proteins.⁽¹⁸⁻²⁰⁾ Lastly, computational experiments have been used to explore RNA-small molecule interactions by docking validated binders into RNA landscapes.⁽²¹⁾

Despite these advances, targeting many RNA targets is still intractable due to limited available data such as 3-dimensional structures, chemotypes that engender RNA binding affinity or selectivity, and RNA structural elements that form small molecule binding sites. Screening of small molecule libraries for binding RNA targets could generate data about the latter two points. However, few small molecule-screening collections are enriched in RNA binders; in fact, most are biased with compounds that bind to proteins.

Myotonic dystrophy (dystrophia myotonica, myotonia atrophica) is a chronic, slowly progressing, highly variable, inherited multisystemic disease. It is characterized by wasting of the muscles (muscular dystrophy), cataracts, heart conduction defects, endocrine changes, and myotonia. Two types of myotonic dystrophy exist. Type 1 (DM1), also known as Steinert disease, has a severe congenital form and a milder childhood-onset form as well as an adult-onset form.

r(CUG) repeating RNA (r(CUG)^{exp}) is implicated in the cause of myotonic dystrophy type 1 (DM1). r(CUG)^{exp} is located in the 3′ untranslated region (UTR) of the dystrophia myotonica protein kinase (DMPK) mRNA.⁽²²⁾ Healthy individuals have 5-37 repeats in the DMPK RNA while DM1-affected individuals have 50-2000 repeats,⁽²³⁾ with disease severity increasing as a function of repeat length.⁽²⁴⁾ r(CUG)^{exp} causes disease via a gain-of-function mechanism in which the RNA binds to and sequesters proteins that are involved in RNA biogenesis.⁽²⁵⁻²⁹⁾ Protein sequestration results in the de-regulation of alternative pre-mRNA splicing⁽²⁶⁻²⁸⁾ and decreased nucleocytoplasmic transport and translation of the DMPK mRNA.³⁰

### SUMMARY OF INVENTION

To address these needs and others, the present invention in various embodiments provides a variety of compounds that bind this RNA and improve disease-associated pre-mRNA splicing defects in cell culture models. Competitive Chemical Cross-Linking and Isolation by Pull Down (C-ChemCLIP) experiments showed that the compounds bind to the intended target in cells. Analysis of the compounds revealed favorable and unfavorable chemotypes that affect bioactivity and selectivity. The RNA-focused compounds are potently bioactive in DM1 cellular assays and they improve DM1 pre-mRNA splicing defects and myotonia in the HSA^{LR} mouse model of DM1.

One embodiment of the invention provides a method for treating myotonic dystrophy type 1 in a human patient suffering therefrom, comprising administering to the patient a therapeutically effective amount of a compound according to formula (I) or a pharmaceutically acceptable salt thereof:

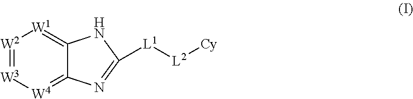

wherein
W¹, W², W³, and W⁴ are independently selected from CR¹ and N, wherein no more than one of W¹, W², W³, and W⁴ is N;
R¹ in each instance is independently selected from the group consisting of H, —NR′R″, —OR′, halo, —C(O)OR′, —C(O)R′, NR′C(O)R″, —NHC(NH)NH₂, —N═CR′((C₆-C₁₀)arylene)R″;
L¹ and L² are independently selected from the group consisting of a bond, (C₁-C₈)alkylene, —NHC(O)—, (C₆-C₁₀)arylene, heteroarylene;
wherein (C₁-C₈)alkylene, (C₆-C₁₀)arylene, and heteroarylene are independently and optionally substituted with one or more substituents selected from —OR′, halo, and —NR′R″;
Cy is selected from the group consisting of (C₆-C₁₀)aryl and heteroaryl;
wherein Cy is optionally substituted with one or more substituents selected from the group consisting of —NR′R″, —OR′, halo, —C(O)OR′, —C(O)R′, —NR′C(O)R″, oxo, —NHC(NH)NH₂; and
R′ and R″ are independently selected from the group consisting of H, (C₁-C₆)alkyl), aryl, heteroaryl.

Another embodiment of the invention is a method for disrupting an RNA-protein complex in a cell, comprising contacting the cell with an effective amount of a compound according to formula (I) as described herein or a pharmaceutically acceptable salt thereof.

In another embodiment, the invention provides a compound according to formula 2GN8:

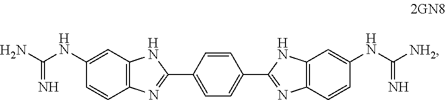 [SMILES: N=C(N)Nc1ccc2nc(-c3ccc(-c4nc5ccc(NC(=N)N)cc5[nH]4)cc3)[nH]c2c1]

or a pharmaceutically acceptable salt thereof.

### BRIEF DESCRIPTION OF THE FIGURES

FIG. 1. The results from screening the RNA-focused small molecule collection for inhibition of the r(CUG)₁₂-MBNL1 complex. (A) In vitro activity of hit compounds for disruption of the r(CUG)₁₂-MBNL1 complex as determined by TR-FRET assay. Compounds were designated as hits if they disrupted the complex at a level ≥3 times the standard deviation of all compounds evaluated. Compounds were screened at 100 μM concentration. (B) Structures of compounds that have demonstrated activity in vitro and in cellulo with IC₅₀ values determined by TR-FRET assay. Compounds 1, 2, and 3 were the most active in vitro while 1, 16, and 17 demonstrated the best activity in cells. (C) Sub-structure analysis of hit compounds identified from the in vitro screen shown in (A). RNA-binding modules identified by the screen include 2-phenyl benzimidazoles, substituted pyridines, and N-phenylbenzamides.

FIG. 2. Bioactivity of hit compounds for improving DM1-associated pre-mRNA splicing defects. (A) Schematic of the pre-mnRNA splicing patterns observed for the cTNT mini-gene in the presence and absence of r(CUG)₉₆₀. (B) Quantification of splicing analysis with representative gel images for the most bioactive compounds, 1, 16 and 17. These compounds were selected because they improve DM1 pre-mRNA splicing defects and do not alter cTNT pre-mRNA splicing in the absence of r(CUG)₉₆₀. Compounds were evaluated at 100 μM concentration. The green line indicates the percentage of exon 5 inclusion observed in healthy, non-DM1 cells while the pink line indicates the percentage of exon 5 inclusion observed in DM1-affected cells. * indicates p<0.05 and ** indicates p<0.01. (C) Dose responses for bioactive hit compounds 16 and 17 for improving cTNT pre-mRNA splicing defects in cells containing r(CUG)₉₆₀.

FIG. 3. Results of competition dialysis experiments of bioactive small molecules with various RNAs and MBNL1. (A) Secondary structures of the RNAs evaluated by competition dialysis. (B) The compounds evaluated selectively bound RNAs containing r(CUG) repeats compared to other RNAs. Targets were tested at 8 μM concentration. Compound 17 had the highest loading on r(CUG)₁₀₉. * indicates p<0.05 and ** indicates p<0.01.

FIG. 4. Target validation of bioactive small molecules. (A) Effects of small molecules on DMPK mRNA levels derived from the DM mini-gene (100 μM concentration). Compounds 16 and 17 had no significant effect on DMPK levels while compound 1 causes a decrease in DMPK levels as determined by a two-tailed Student t-test. *** indicates p<0.001 (B) Schematics of Chemical Cross-Linking and Isolation by Pull-down (ChemCLIP) and Competitive ChemCLIP (C-ChemCLIP). Cells containing r(CUG)₉₆₀ are co-treated with a small molecule known to bind and cross-link to r(CUG)^{exp} in cellulo, 2H-4-CA-Biotin, and a bioactive small molecule. Following affinity purification, in cellulo binding to the target RNA is assessed by qRT-PCR. (C) Results of C-ChemCLIP with bioactive small molecules 1, 16, and 17. These compounds bound r(CUG)₉₆₀ to a similar extent in cells.

FIG. 5. Synthesis of 2GN8.

FIG. 6. Results of competition dialysis experiments with 2GN8 and various RNAs and MBNL1. 2GN8 selectively bound RNAs containing r(CUG) repeats compared to other RNAs. Targets were tested at 8 μM concentration.

FIG. 7. Results of qPCR experiments showing 2GN8 does not alter the level of DMPK mRNA in patient derived fibroblasts.

FIG. 8. Results of C-ChemCLIP experiments with 2GN8 indicating that r(CUG)₉₆₀ is the target of 2GN8 in transfected cells.

FIG. 9. 2GN8 improves r(CUG)^{exp}-MBNL1 translational defects in a cell-based luciferase reporter assay. (A) Schematic of the cell model used to study translational defects. A stably transfected C2C12 cell line expresses firefly luciferase mRNA with r(CUG)₈₀₀ in the 3′ UTR. r(CUG)₈₀₀ causes the transcript to be mostly retained in the nucleus and thus not efficiently translated. If a small molecule binds to r(CUG)₈₀₀ and displaces or inhibits MBNL1 binding, then the transcript is more efficiently exported from the nucleus and translated in the cytoplasm. (B) 2GN8 improves translational defects at 300 and 200 μM while demonstrating no off target effects in cells lacking r(CUG)₈₀₀.

FIG. 10. 2GN8 improves r(CUG)^{exp}-MBNL1 pre-mRNA splicing defects in patient derived fibroblasts containing 500 CUG repeats.

FIG. 11. 2GN8 reduces r(CUG)^{exp}-MBNL1 associated nuclear foci in patient derived fibroblasts containing 500 CUG repeats.

FIG. 12. Evaluation of 2GN8 DM1 HSA^{LR} mice. (A) Schematic of Clcn1 and Serca1 pre-mRNA splicing defects. (B) After 18 days of treatment, pre-mRNA splicing defects are improved when treated with 40 mg/kg/day of 2GN8.

FIG. 13. 2GN8 improves myotonia grade in DM1 HSA^{LR} mice after treating with 40 mg/kg/day.

### DETAILED DESCRIPTION

### Definitions

“Alkyl” refers to straight, branched chain, or cyclic hydrocarbyl groups including from 1 to about 20 carbon atoms. For instance, an alkyl can have from 1 to 10 carbon atoms or 1 to 5 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —C(CH₃)(CH₂CH₃), —CH(CH₂CH₃)₂, —C(CH₃)₃, —C(CH₂CH₃)₃, —CH₂CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃)CH(CH₃)(CH₂CH₃), —CH₂CH₂CH(CH₂)₂, —CH₂CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃)CH₂CH(CH₃)₂, —CH(CH₃)CH(CH₃)CH(CH₃)₂, and the like. Thus, alkyl groups include primary alkyl groups, secondary alkyl groups, and tertiary alkyl groups.

The phrase “substituted alkyl” refers to alkyl substituted at 1 or more, e.g., 1, 2, 3, 4, 5, or even 6 positions, which substituents are attached at any available atom to produce a stable compound, with substitution as described herein. “Optionally substituted alkyl” refers to alkyl or substituted alkyl.

Each of the terms “halogen,” “halide,” and “halo” refers to —F, —Cl, —Br, or —I.

The terms “alkylene” and “substituted alkylene” refer to divalent alkyl and divalent substituted alkyl, respectively. Examples of alkylene include without limitation, ethylene (—CH₂—CH₂—). “Optionally substituted alkylene” refers to alkylene or substituted alkylene.

“Alkene” refers to straight, branched chain, or cyclic hydrocarbyl groups including from 2 to about 20 carbon atoms having one or more carbon to carbon double bonds, such as 1 to 3, 1 to 2, or at least one carbon to carbon double bond. “Substituted alkene” refers to alkene substituted at 1 or more, e.g., 1, 2, 3, 4, 5, or even 6 positions, which substituents are attached at any available atom to produce a stable compound, with substitution as described herein. “Optionally substituted alkene” refers to alkene or substituted alkene.

The term “alkenylene” refers to divalent alkene. Examples of alkenylene include without limitation, ethenylene (—CH═CH—) and all stereoisomeric and conformational isomeric forms thereof. “Substituted alkenylene” refers to divalent substituted alkene. “Optionally substituted alkenylene” refers to alkenylene or substituted alkenylene.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein below.

The term “alkynylene” refers to divalent alkyne. Examples of alkynylene include without limitation, ethynylene, propynylene. “Substituted alkynylene” refers to divalent substituted alkyne.

The term “alkoxy” refers to an —O-alkyl group having the indicated number of carbon atoms. For example, a (C₁-C₆)alkoxy group includes —O-methyl (methoxy), —O-ethyl (ethoxy), —O-propyl (propoxy), —O-isopropyl (isopropoxy), —O-butyl (butoxy), —O-sec-butyl (sec-butoxy), —O-tert-butyl (tert-butoxy), —O-pentyl (pentoxy), —O-isopentyl (isopentoxy), —O-neopentyl (neopentoxy), —O-hexyl (hexyloxy), —O-isohexyl (isohexyloxy), and —O-neohexyl (neohexyloxy).

The term “aryl,” alone or in combination refers to an aromatic monocyclic or bicyclic ring system such as phenyl or naphthyl. “Aryl” also includes aromatic ring systems that are optionally fused with a cycloalkyl ring as herein defined.

A “substituted aryl” is an aryl that is independently substituted with one or more substituents attached at any available atom to produce a stable compound, wherein the substituents are as described herein. “Optionally substituted aryl” refers to aril or substituted aryl.

“Arylene” denotes divalent aryl, and “substituted arylene” refers to divalent substituted aryl. “Optionally substituted arylene” refers to arylene or substituted arylene.

The term “heteroatom” refers to N, O, and S. Inventive compounds that contain N or S atoms can be optionally oxidized to the corresponding N-oxide, sulfoxide or sulfone compounds.

“Heteroaryl,” alone or in combination with any other moiety described herein, refers to a monocyclic aromatic ring structure containing 5 or 6 ring atoms, or a bicyclic aromatic group having 8 to 10 atoms, containing one or more, such as 1 to 4, 1 to 3, or 1 to 2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinoxalyl, indolizinyl, benzol bithienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. “Heteroaryl” also contemplates fused ring systems wherein the heteroaryl is fused to an aryl or cycloalkyl ring as defined herein.

A “substituted heteroaryl” is a heteroaryl that is independently substituted, unless indicated otherwise, with one or more, e.g., 1, 2, 3, 4 or 5, attached at any available atom to produce a stable compound, wherein the substituents are as described herein. “Optionally substituted heteroaryl” refers to heteroaryl or substituted heteroaryl.

“Heteroarylene” refers to divalent heteroaryl, and “substituted heteroarylene” refers to divalent substituted heteroaryl. “Optionally substituted heteroarylene” refers to heteroarylene or substituted heteroarylene.

“Heterocycloalkyl” means a saturated or unsaturated non-aromatic monocyclic, bicyclic, tricyclic or polycyclic ring system that has from 5 to 14 atoms in which from 1 to 3 carbon atoms in the ring are replaced by heteroatoms of O, S or N. A heterocycloalkyl is optionally fused with benzo or heteroaryl of 5-6 ring members, and includes oxidized S or N, such as sulfinyl, sulfonyl and N-oxide of a tertiary ring nitrogen. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl.

“Optionally substituted heterocycloalkyl” denotes heterocycloalkyl that is substituted with to 3 substituents, e.g., 1, 2 or 3 substituents, attached at any available atom to produce a stable compound, wherein the substituents are as described herein.

“Heteroalkyl” means a saturated alkyl group having from 1 to about 20 carbon atoms, 1 to 10 carbon atoms, 1 to 6 carbon atoms, or 1 to 3 carbon atoms, in which from 1 to 3 carbon atoms are replaced by heteroatoms of O, S or N. Heteroalkyl is also intended to include oxidized S or N, such as sulfinyl, sulfonyl and N-oxide of a tertiary ring nitrogen. The point of attachment of the heteroalkyl substituent is at an atom such that a stable compound is formed. Examples of heteroalkyl groups include, but are not limited to, N-alkylaminoalkyl (e.g., CH₃NHCH₂—), N,N-dialkylaminoalkyl (e.g., (CH₃)₂NCH₂—), and the like.

“Heteroalkylene” refers to divalent heteroalkyl. The term “optionally substituted heteroalkylene” refers to heteroalkylene that is substituted with 1 to 3 substituents, e.g., 1, 2 or 3 substituents, attached at any available atom to produce a stable compound, wherein the substituents are as described herein.

“Heteroalkene” means a unsaturated alkyl group having from 1 to about 20 carbon atoms, 1 to 10 carbon atoms, 1 to 6 carbon atoms, or 1 to 3 carbon atoms, in which from 1 to 3 carbon atoms are replaced by heteroatoms of O, S or N, and having 1 to 3, 1 to 2, or at least one carbon to carbon double bond or carbon to heteroatom double bond.

“Heteroalkenylene” refers to divalent heteroalkene. The term “optionally substituted heteroalkenylene” refers to heteroalkenylene that is substituted with 1 to 3 substituents, e.g., 1, 2 or 3 substituents, attached at any available atom to produce a stable compound, wherein the substituents are as described herein.

The term “cycloalkyl” refer to monocyclic, bicyclic, tricyclic, or polycyclic, 3- to 14-membered ring systems, which are either saturated, unsaturated or aromatic. The cycloalkyl group may be attached via any atom. Cycloalkyl also contemplates fused rings wherein the cycloalkyl is fused to an aryl or hetroaryl ring as defined above. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein below.

The term “cycloalkenyl” refers to a monocyclic, bicyclic, tricyclic, or polycyclic, 3- to 14-membered ring system, which is unsaturated. The cycloalkenyl group may be attached via any atom. Representative examples of cycloalkenyl include, but are not limited to, cyclopropenyl, cyclobutenyl, cyclopentenyl and cyclohexenyl.

The term “cycloalkylene” refers to divalent cycloalkyl. The term “optionally substituted cycloalkylene” refers to cycloalkylene that is substituted with 1 to 3 substituents, e.g., 1, 2 or 3 substituents, attached at any available atom to produce a stable compound, wherein the substituents are as described herein.

The term “nitrile or cyano” can be used interchangeably and refer to a —CN group which is bound to a carbon atom of a heteroaryl ring, aryl ring and a heterocycloalkyl ring.

The term “oxo” refers to a ═O atom attached to a saturated or unsaturated (C₃-C₈) cyclic or a (C₁-C₈) acyclic moiety. The ═O atom can be attached to a carbon, sulfur, and nitrogen atom that is part of the cyclic or acyclic moiety.

The term “amine or amino” refers to an —NR^{d}R^{e} group wherein R^{d} and R^{e} each independently refer to a hydrogen, (C₁-C₈)alkyl, aryl, heteroaryl, heterocycloalkyl, (C₁-C₈)haloalkyl, and (C₁-C₆)hydroxyalkyl group.

The term “amide” refers to a —NR′R″C(O)— group wherein R′ and R″ each independently refer to a hydrogen, (C₁-C₈)alkyl, or (C₃-C₆)aryl.

The term “carboxamido” refers to a —C(O)NR′R″ group wherein R′ and R″ each independently refer to a hydrogen, (C₁-C₈)alkyl, or (C₃-C₆)aryl.

The term “aryloxy” refers to an —O-aryl group having the indicated number of carbon atoms. Examples of aryloxy groups include, but are not limited to, phenoxy, naphthoxy and cyclopropeneoxy.

The term “haloalkoxy,” refers to an —O—(C₁-C₆)alkyl group wherein one or more hydrogen atoms in the C₁-C₈ alkyl group is replaced with a halogen atom, which can be the same or different. Examples of haloalkyl groups include, but are not limited to, difluoromethoxy, trifluoromethoxy, 2,2,2-trifluoroethoxy, 4-chlorobutoxy, 3-bromopropyloxy, pentachloroethoxy, and 1,1,1-trifluoro-2-bromo-2-chloroethoxy.

The term “hydroxyalkyl,” refers to an alkyl group having the indicated number of carbon atoms wherein one or more of the alkyl group's hydrogen atoms is replaced with an —OH group. Examples of hydroxyalkyl groups include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH₂CH₂CH₂CH₂CH₂OH, and branched versions thereof.

The term “alkylsulfonyl” refers to a (C₁-C₆)alkyl group wherein one or more hydrogen atoms in the C₁-C₆ alkyl group is replaced with a —S(O)ₐ group. Subscript “a” can either be 1 or 2, so as to give an alkyl sulfoxide (sulfinyl group), or an alkyl sulfone respectively. Examples of alkylsulfonyl groups include, but are not limited to dimethylsulfoxide, ethyl methyl sulfoxide, and methylvinylsulfone.

The term “haloalkyl,” refers to an (C₁-C₆)alkyl group wherein one or more hydrogen atoms in the C₁-C₆ alkyl group is replaced with a halogen atom, which can be the same or different. Examples of haloalkyl groups include, but are not limited to, difluoromethyl, trifluoromethyl, 2,2,2-trifluoroethyl, 4-chlorobutyl, 3-bromopropylyl, pentachloroethyl, and 1,1,1-trifluoro-2-bromo-2-chloroethyl.

The term “aminoalkyl,” refers to an (C₁-C₆)alkyl group wherein one or more hydrogen atoms in the C₁-C₆ alkyl group is replaced with a —NR^{d}R^{e} group, where R^{d} and R^{e} can be the same or different, for example, R^{d} and R^{e} each independently refer to a hydrogen, (C₁-C₈)alkyl, aryl, heteroaryl, heterocycloalkyl (C₁-C₈)haloalkyl, and (C₁-C₆)hydroxyalkyl group. Examples of aminoalkyl groups include, but are not limited to, aminomethyl, aminoethyl, 4-aminobutyl and 3-aminobutylyl.

The term “thioalkyl” or “alkylthio” refers to a (C₁-C₆)alkyl group wherein one or more hydrogen atoms in the C₁-C₆ alkyl group is replaced with a —SR^{j} group, wherein R^{j} is selected from the group consisting of hydrogen, (C₁-C₆)alkyl and (C₃-C₁₄)aryl.

“Amino (C₁-C₆)alkylene” refers to a divalent alkylene wherein one or more hydrogen atoms in the C₁-C₆ alkylene group is replaced with a —NR^{d}R^{e} group. Examples of amino (C₁-C₆)alkylene include, but are not limited to, aminomethylene, aminoethylene, 4-aminobutylene and 3-aminobutylylene.

The term “sulfonamide” refers to an —NR^{g}S(O)₂R^{h} group where R^{g} and R^{h} are each independently refer to a hydrogen, (C₁-C₈)alkyl, aryl, heteroaryl, heterocycloalkyl, (C₁-C₈)haloalkyl, and (C₁-C₆)hydroxyalkyl group.

A “hydroxyl” or “hydroxy” refers to an —OH group.

The term “(C₃-C₁₄)aryl-(C₁-C₆)alkylene” refers to a divalent alkylene wherein one or more hydrogen atoms in the C₁-C₆ alkylene group is replaced by a (C₃-C₁₄)aryl group. Examples of (C₃-C₁₄)aryl-(C₁-C₆)alkylene groups include without limitation 1-phenylbutylene, phenyl-2-butylene, 1-phenyl-2-methylpropylene, phenylmethylene, phenylpropylene, and naphthylethylene.

The term “(C₃-C₁₄)heteroaryl-(C₁-C₆)alkylene” refers to a divalent alkylene wherein one or more hydrogen atoms in the C₁-C₆ alkylene group is replaced a (C₃-C₁₄)heteroaryl group. Examples of (C₃-C₄)heteroaryl-(C₁-C₆)alkylene groups include without limitation 1-pyridylbutylene, quinolinyl-2-butylene and 1-pyridyl-2-methylpropylene.

The term “(C₃-C₁₄)heterocycloalkyl-(C₁-C₆)alkylene” refers to a divalent alkylene wherein one or more hydrogen atoms in the C₁-C₆ alkylene group is replaced by a (C₃-C₁₄)heterocycloalkyl group. Examples of (C₃-C₁₄)heterocycloalkyl-(C₁-C₆)alkylene groups include without limitation 1-morpholinopropylene, azetidinyl-2-butylene and 1-tetrahydrofuranyl-2-methylpropylene.

The term “(C₃-C₁₄)heteroaryl-(C₁-C₁₄)hetercycloalkylene” refers to a divalent heterocycloalkylene wherein one or more hydrogen atoms in the C₁-C₆ heterocycloalkylene group is replaced by a (C₃-C₁₄)heteroaryl group. Examples of (C₃-C₁₄)heteroaryl-(C₁-C₆)heterocycloalkylene groups include without limitation pyridylazetidinylene and 4-quinolino-1-piperazinylene.

The term “(C₃-C₁₄)aryl-(C₁-C₁₄)heterocycloalkylene” refers to a divalent heterocycloalkyl ene wherein one or more hydrogen atoms in the C₁-C₁₄ heterocycloalkylene group is replaced by a (C₃-C₁₄)aryl group. Examples of (C₃-C₁₄)aryl-(C₁-C₁₄)heterocycloalkylene groups include without limitation 1-naphthyl-piperazinylene, phenylazetidinylene, and phenylpiperidinylene.

The term “(C₃-C₁₄)aryl-(C₁-C₆)alkyl-(C₁-C₁₄)heterocycloalkylene” refers to a divalent heterocycloalkylene wherein one or more hydrogen atoms in the C₁-C₁₄ heterocycloalkylene group is replaced by a (C₁-C₆) alkyl group that is further substituted by replacing one or more hydrogen atoms of the (C₁-C₆) alkyl group with a (C₃-C₁₄)aryl group.

The term “(C₃-C₁₄)heteroaryl-(C₁-C₆)alkyl-(C₁-C₁₄)heterocycloalkylene” refers to a divalent heterocycloalkylene wherein one or more hydrogen atoms in the C₁-C₁₄ heterocycloalkylene group is replaced by a (C₁-C₆) alkyl group that is further substituted by replacing one or more hydrogen atoms of the (C₁-C₆) alkyl group with a (C₃-C₁₄)heteroaryl group.

The term “(C₃-C₁₄)heterocycloalkyl-(C₁-C₆)alkyl-(C₁-C₁₄)heterocycloalkyl ene” refers to a divalent heterocycloalkylene wherein one or more hydrogen atoms in the C₁-C₁₄ heterocycloalkylene group is replaced by a (C₁-C₆) alkyl group that is further substituted by replacing one or more hydrogen atoms of the (C₁-C₆) alkyl group with a (C₃-C₁₄)heterocycloalkyl group.

The term “(C₃-C₁₄)aryl-(C₁-C₁₄)cycloalkylene” refers to a divalent cycloalkylene that is monocyclic, bicyclic or polycyclic and wherein one or more hydrogen atoms in the (C₁-C₁₄)cycloalkylene group is replaced by a (C₃-C₁₄)aryl group. Examples of (C₃-C₁₄)aryl-(C₁-C₁₄)cycloalkylene groups include without limitation phenylcyclobutylene, phenyl-cyclopropylene and 3-phenyl-2-methylbutylene-1-one.

The substituent —CO₂H may be replaced with bioisosteric replacements such as:

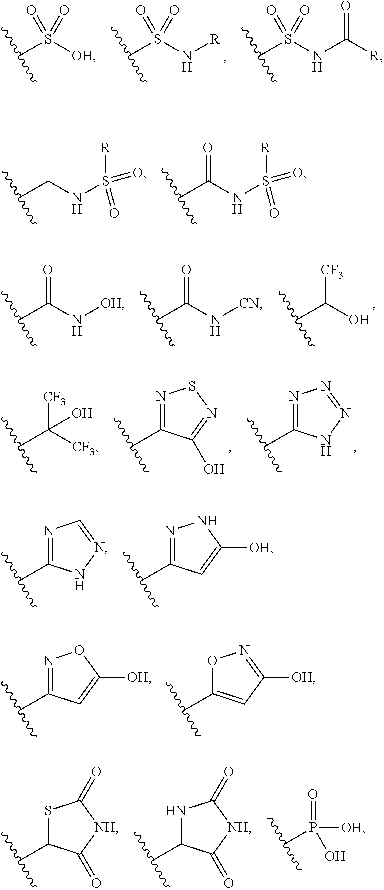

and the like, wherein R has the same definition as R′ and R″ as defined herein. See, e.g., THE PRACTICE OF MEDICINAL CHEMISTRY (Academic Press: New York, 1996), at page 203.

The compound of the invention can exist in various isomeric forms, including configurational, geometric, and conformational isomers, including, for example, cis- or trans-conformations. Compounds of the present invention may also exist in one or more tautomeric forms, including both single tautomers and mixtures of tautomers. The term “isomer” is intended to encompass all isomeric forms of a compound of this invention, including tautomeric forms of the compound.

Some compounds described here can have asymmetric centers and therefore exist in different enantiomeric and diastereomeric forms. A compound of the invention can be in the form of an optical isomer or a diastereomer. Accordingly, the invention encompasses compounds of the invention and their uses as described herein in the form of their optical isomers, diastereoisomers and mixtures thereof, including a racemic mixture. Optical isomers of the compounds of the invention can be obtained by known techniques such as asymmetric synthesis, chiral chromatography, simulated moving bed technology or via chemical separation of stereoisomers through the employment of optically active resolving agents.

Unless otherwise indicated, “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiorners to help describe the relative stereochemistry. Those skilled in the art of organic synthesis will know if the compounds are prepared as single enantiomers from the methods used to prepare them.

A “pharmaceutically acceptable salt” is a pharmaceutically acceptable, organic or inorganic acid or base salt of a compound of the invention. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In certain embodiments, such terms refer to minimizing the spread or worsening of the disease resulting from the administration of one or more prophylactic or therapeutic agents to a patient with such a disease.

The terms “prevent,” “preventing,” and “prevention” refer to the prevention of the onset, recurrence, or spread of the disease in a patient resulting from the administration of a prophylactic or therapeutic agent.

The term “effective amount” refers to an amount of a compound of the invention, such as a Formula I compound, or other active ingredient sufficient to provide a therapeutic or prophylactic benefit in the treatment or prevention of a disease or to delay or minimize symptoms associated with a disease. Further, a therapeutically effective amount with respect to a compound of the invention means that amount of therapeutic agent alone, or in combination with other therapies, that provides a therapeutic benefit in the treatment or prevention of a disease. Used in connection with a compound of the invention, the term can encompass an amount that improves overall therapy, reduces or avoids symptoms or causes of disease, or enhances the therapeutic efficacy of or synergies with another therapeutic agent.

A “patient” includes an animal, such as a human, cow, horse, sheep, lamb, pig, chicken, turkey, quail, cat, dog, mouse, rat, rabbit or guinea pig. The animal can be a mammal such as a non-primate and a primate (e.g., monkey and human). In one embodiment, a patient is a human, such as a human infant, child, adolescent or adult.

In Vitro Screening of Compounds for Disruption of an RNA-Protein Complex that Causes DM1.

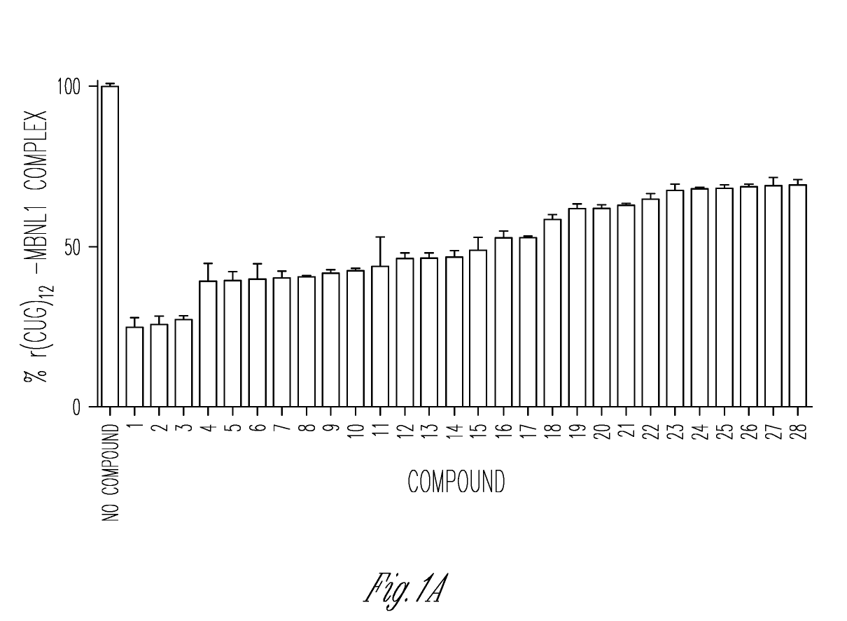

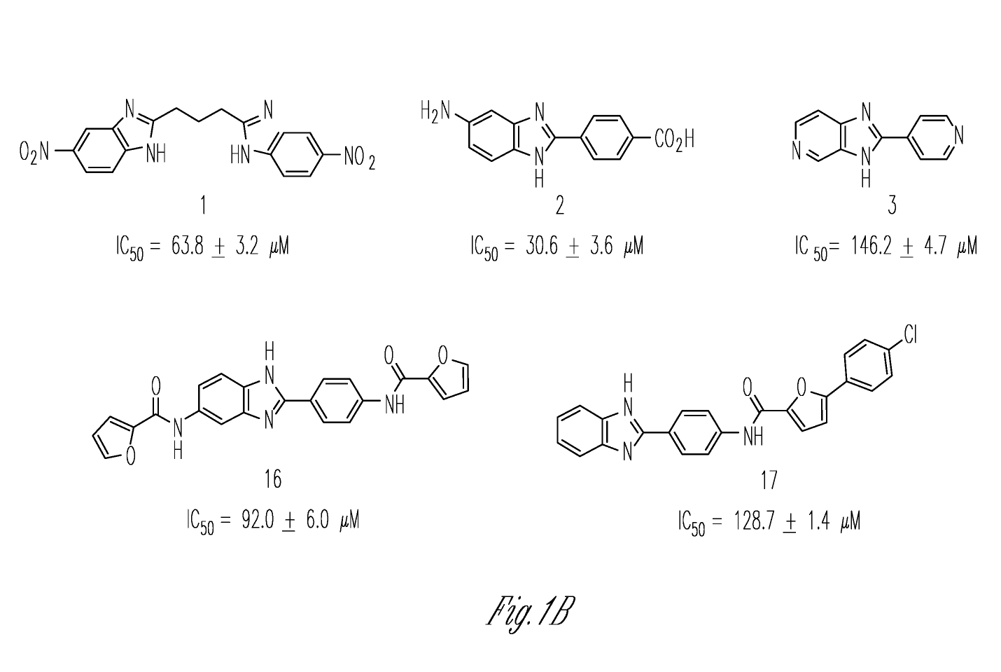

DM1 is caused by an RNA gain-of-function mechanism in which r(CUG)^{exp} binds and inactivates proteins involved RNA biogenesis.⁽²⁵⁻²⁹⁾ One protein, muscleblind-like 1 (MBNL1), regulates the alternative splicing of a sub-set of RNAs that are de-regulated in DM1.^{(27, 43-45)} We discovered that Formula (I) compounds disrupt the r(CUG)₁₂-MBNL1 complex in vitro as determined by a previously described time-resolved fluorescence resonance energy transfer (TR-FRET) assay (FIG. 1A).⁽⁴⁶⁾

Exemplary Formula (I) compounds satisfying this assay, among other criteria, include the following compounds:

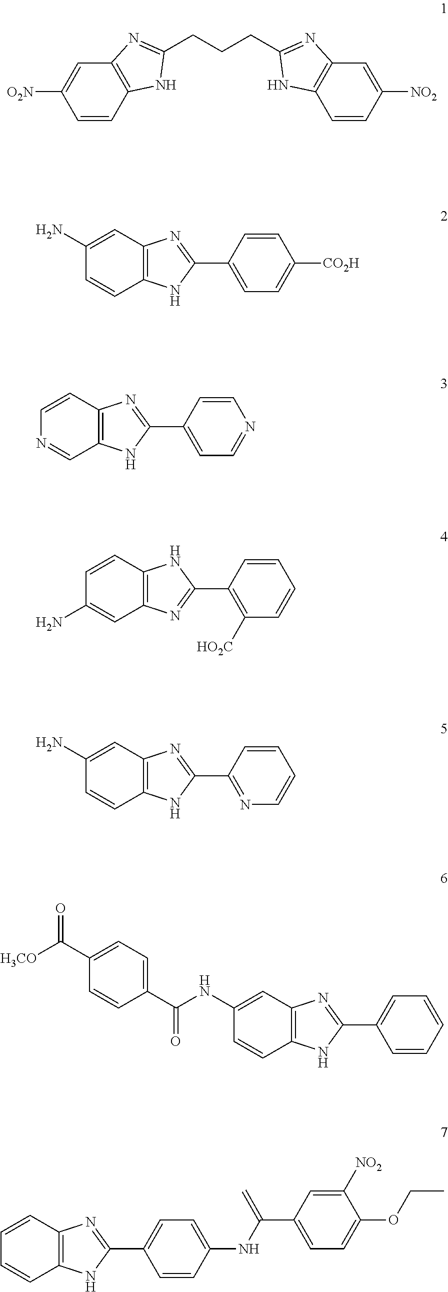

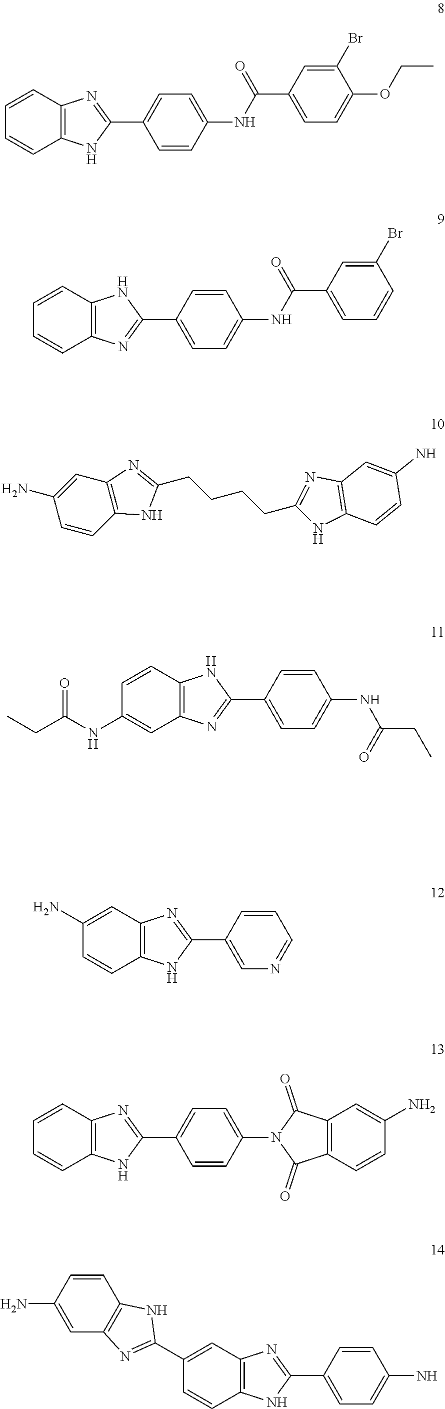

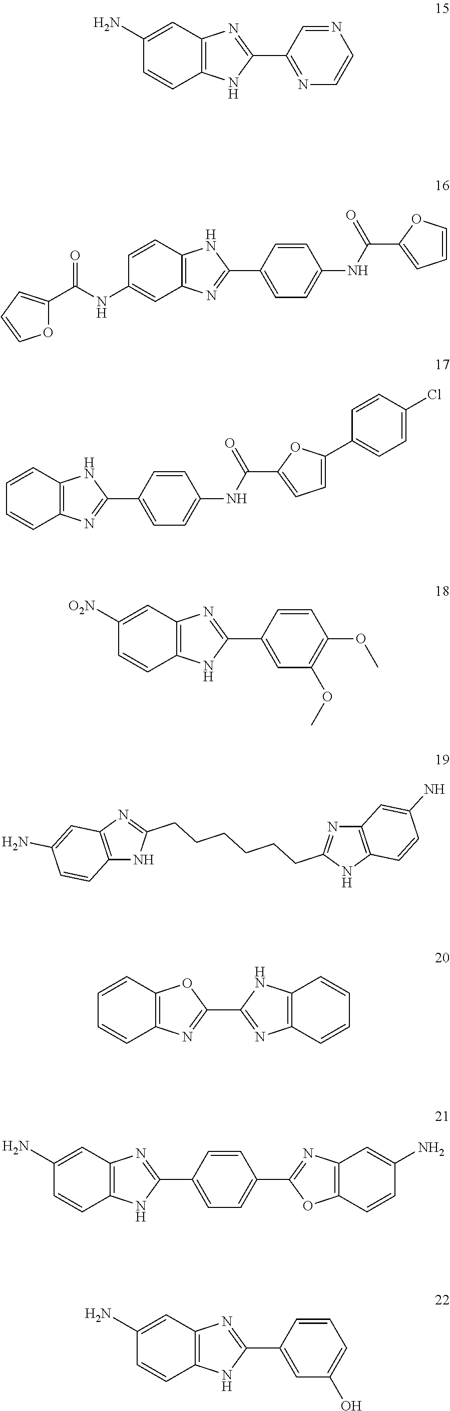

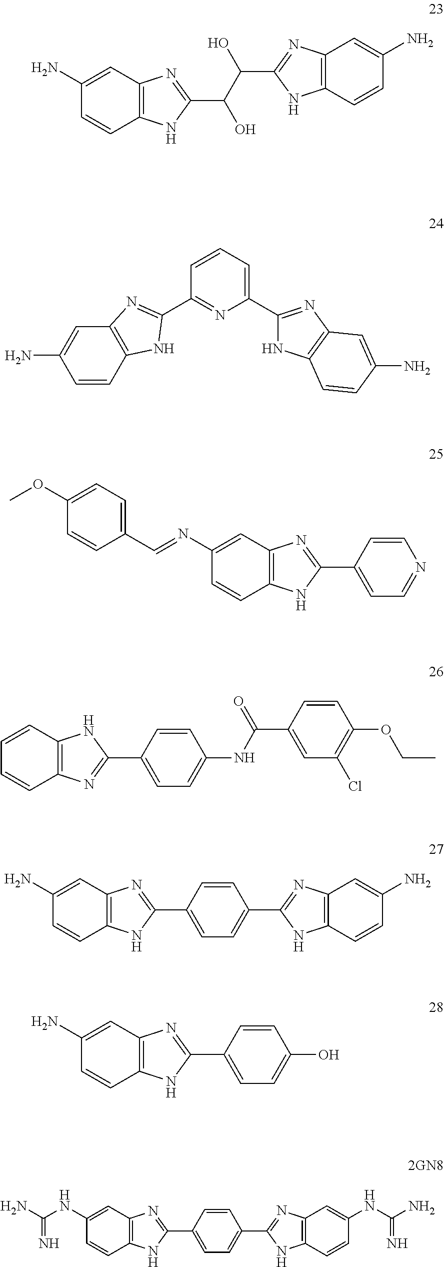

The most potent inhibitors disrupted >70% of the complex at 100 μM concentration; these included compounds 1, 2, and 3 (FIG. 1B). In general, Formula (I) compounds inhibited ≥50% of the complex at this concentration IC₅₀ analysis of select compounds was completed and the results are shown in FIG. 1B. The IC₅₀ values for these compounds range from 30-˜150 μM. Typically, lead compounds contained multiple sub-structures that can interact with RNA.⁽³⁵⁾

Exemplary compounds were then subjected to sub-structure analysis as described previously.^{(32, 35)} The most common sub-structures contained substituted pyridyl, benzimidazole, or imidazole ring systems. Specific functionalities derived from these sub-structures include benzyl imidazoles, benzyl benzimidazoles, and pyridyl benzimidazoles; although they occur ˜2 times more frequently in hit compounds than in a 320-member library, they are not statistically significant (FIG. 1C). These chemotypes have been found in other RNA binders and have comprised structures in RNA-focused compound collections.⁽³⁵⁾

### Bioactivity: Improving DM1-Associated Alternative Pre-mRNA Splicing Defects.

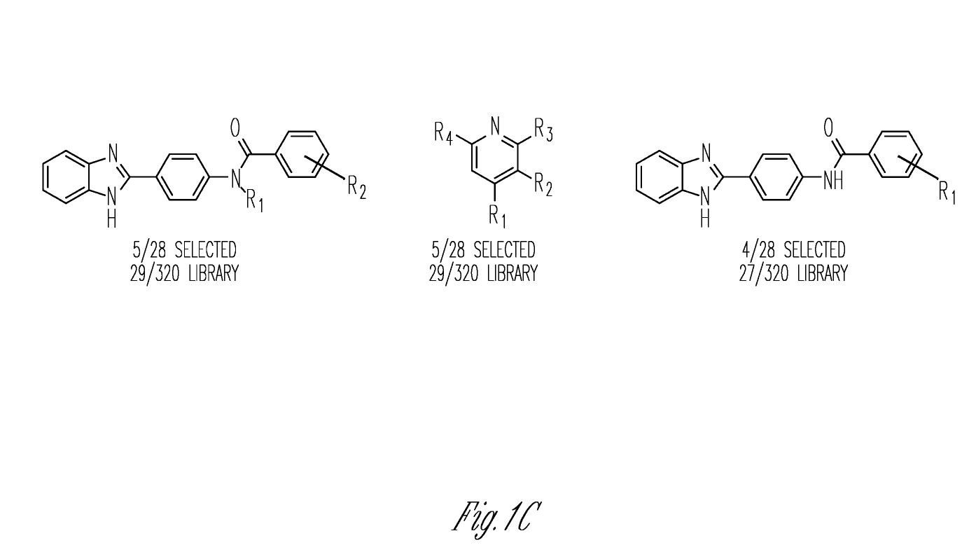

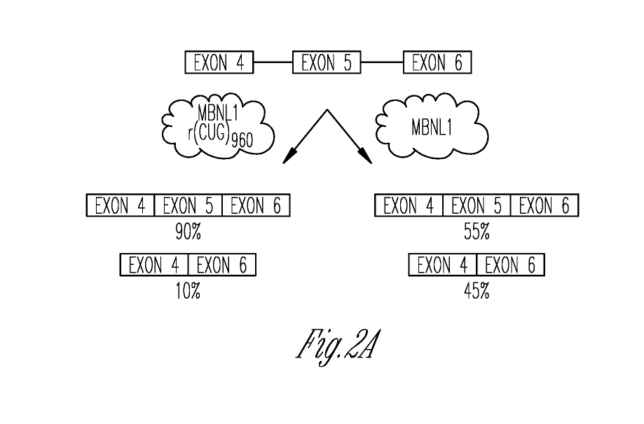

The biological activity of Formula (I) compounds was studied in a DM1 cellular model. Specifically, Formula (I) compounds were tested for their ability to improve DM1-associated pre-mRNA splicing defects caused by sequestration of MBNL1 (FIG. 2).⁽⁴⁷⁾ In particular, we co-transfected a DM1 mini-gene that contains 960 r(CUG) repeats and a mini-gene that reports on alternative splicing of cardiac troponin T (cTNT) exon 5, which is de-regulated in DM1.²⁵ In the presence of r(CUG)₉₆₀, ˜90% of exon 5 is included in the mature mRNA while the amount of mature mRNA containing exon 5 in healthy cells is ˜55% (absence of r(CUG)₉₆₀) (FIGS. 2A & 2B). According to this standard, if a compound targets r(CUG)₉₆₀ and frees MBNL1 in cellulo, then a decrease in exon 5 inclusion should be observed. In these experiments, exemplary Formula (I) compounds improved cTNT alternative pre-mRNA splicing patterns toward wild type (FIG. 2B).

The specificity of the compounds was then assessed by monitoring changes in cTNT splicing patterns in cells that do not express r(CUG)₉₆₀. Compounds that are selective for the DM1 RNA should have no effect on cTNT pre-mRNA splicing in healthy cells that do not express r(CUG)₉₆₀. Based on potency and selectivity according to these standards, exemplary Formula (I) compounds were determined to include 1, 16, and 17 (FIGS. 1B & 2). These three compounds do not have a positive charge at physiological pH. One goal of drug discovery efforts for RNA targets is to find uncharged, bioactive compounds. These molecules appear to meet this goal, vide infra.

Compounds 16 and 17 were evaluated by dose response and these compounds retained modest activity at 10 μM for improving cTNT pre-mRNA splicing defects (FIG. 2C). The toxicity of compounds 1, 16 and 17 was assessed in this cellular system by using a WST-1 assay and the compounds do not affect cell viability. The selectivities of these molecules were further studied in vitro by competition dialysis and in cellulo for target engagement using a method developed in our laboratory called Chemical Cross-Linking and Isolation by Pull-down (ChemCLIP).^{(48, 49)}

### In Vitro Selectivity of Bioactive Small Molecules 1, 16, and 17.

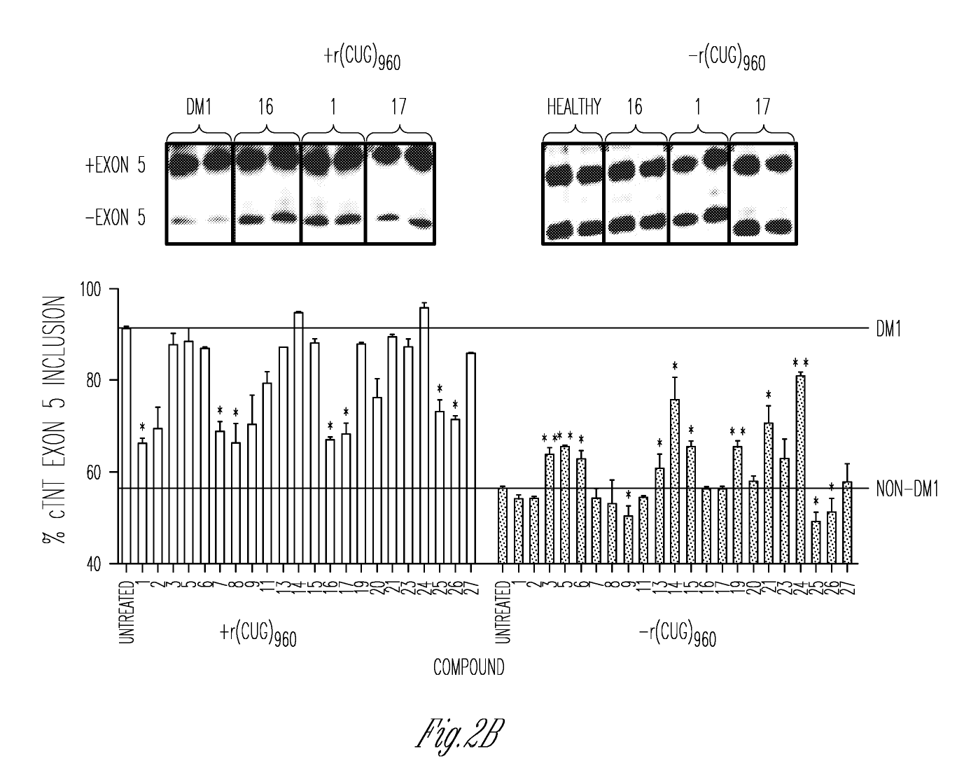

The selectivities of compounds 1, 16, and 17 were measured by competition dialysis using various RNA and protein targets. RNA targets included a mimic of the human rRNA A-site, the MBNL1-binding site in the cTNT pre-mRNA, an RNA with GC base pairs, and RNAs with different numbers of r(CUG) repeats (r(CUG)_{1×2}, r(CUG)_{6×2}, and r(CUG)₁₀₉, an expanded repeat of a length causative of disease⁽²³⁾) (FIG. 3A). By studying these RNAs, we gained insight into compound selectivity by determining whether a compound binds cooperatively to RNAs with multiple 5′CUG/3′GUC motifs (secondary structure displayed by r(CUG)^{exp}; FIG. 3A). Binding to MBNL1 was also evaluated to assess the propensity of the compounds to bind MBNL1 over r(CUG)^{exp}.

All three exemplary Formula (I) compounds bind the desired target, r(CUG)₁₀₉, to the greatest extent as compared to the other RNAs and MBNL1. The Formula (I) compounds with the highest loading onto r(CUG)₁₀₉ was also the most bioactive, compound 17. Although r(CUG)₁₀₉ has ˜9 times more 5′CUG/3′GUC binding sites than r(CUG)_{6×2}, the loading of 17 is ˜14-fold higher on r(CUG)₁₀₉ than r(CUG)_{6×2}. This suggests that 17 binds cooperatively to RNAs with longer repeats, which may be an important factor for target recognition and bioactivity.

It has been previously observed that compounds can bind to r(CUG)^{exp} with positive cooperativity.⁽⁵⁰⁾ Compound 16 may not benefit from cooperative binding as it has the lowest loading onto r(CUG)₁₀₉ yet the compound still selectively improves DM1 pre-mRNA splicing defects. This effect likely becomes significant as the repeat length increases. In this in vitro screen, the repeat length is 12 while the pathogenic repeat length used in cellular studies is 960.

### In Cellulo Selectivity of Formula (I) Compound Nos. 1, 16, and 17.

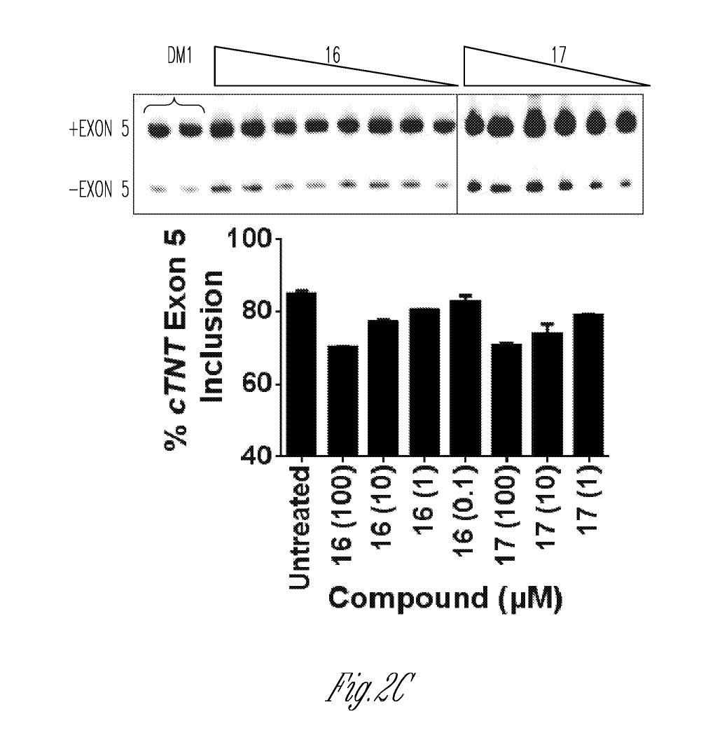

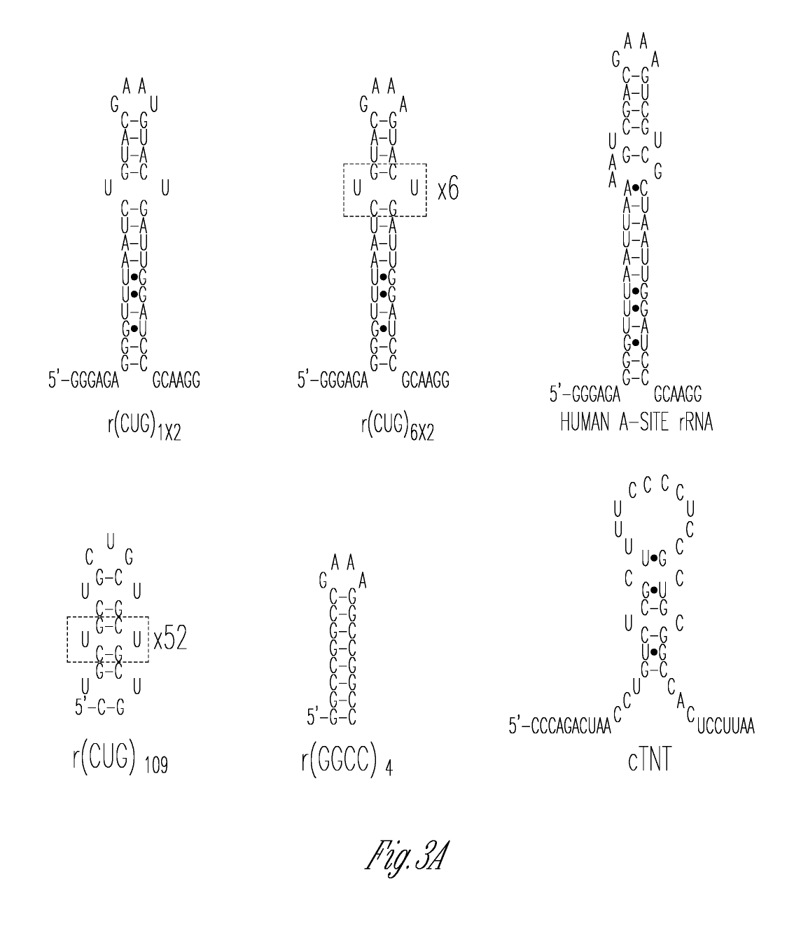

In some previously reported studies, small molecules improve DM1-associated defects not by direct target engagement but rather by decreasing levels of r(CUG)^{exp} (DMPK) at the transcriptional level.⁽⁵¹⁾ Therefore, to validate the mechanism of action for the compounds described herein, we studied if 1, 16, and 17 bind r(CUG)^{exp} in cellulo as well as their effects on the abundance of DMPK mRNA (FIG. 4). At bioactive concentrations, neither 16 nor 17 had any effect on the abundance of DMPK mRNA while compound 1 diminished DMPK abundance by ˜40% (FIG. 4A).

The biological targets of Formula (I) compounds were further investigated by using Competitive ChemCLIP (C-ChemCLIP) (FIG. 4B).(48, 49) In ChemCLIP, an RNA-binding compound is appended with: (i) a reactive module (chlorambucil; CA) that cross-links to the RNAs that the small molecule binds in cellulo; and (ii) a biotin moiety for facile pull-down and isolation of those targets.⁽⁴⁹⁾ The pulled-down fraction is then analyzed by qRT-PCR. In this particular application, we used C-ChemCLIP using a previously studied compound, 2H-4-CA-Biotin, that binds and cross-links to r(CUG)^{exp} in cellulo.⁽⁴⁹⁾ If exemplary Formula (I) compounds also engage r(CUG)^{exp} in cellulo, then the amount of the RNA pulled down by 2H-4-CA-Biotin should decrease as a function of 1, 16, or 17 concentration. In the presence of 1, 16 and 17 the amount of r(CUG)^{exp} pulled down by 2H-4-CA-Biotin is significantly reduced and indicates that the small molecules bind r(CUG)^{exp} (FIG. 4C). Taken together, the bioactivity of 16 and 17 can be directly attributed to binding r(CUG)^{exp}; the bioactivity of compound 1 may have a mixed mode of inhibition in which the compound inhibits transcription of the r(CUG)^{exp}-containing mRNA or causes its degradation and binds the repeats, all of which could lead to improvement of r(CUG)^{exp}-associated dysfunction.

### Optimization of Formula (I) Compounds by Utilizing Medicinal Chemistry.

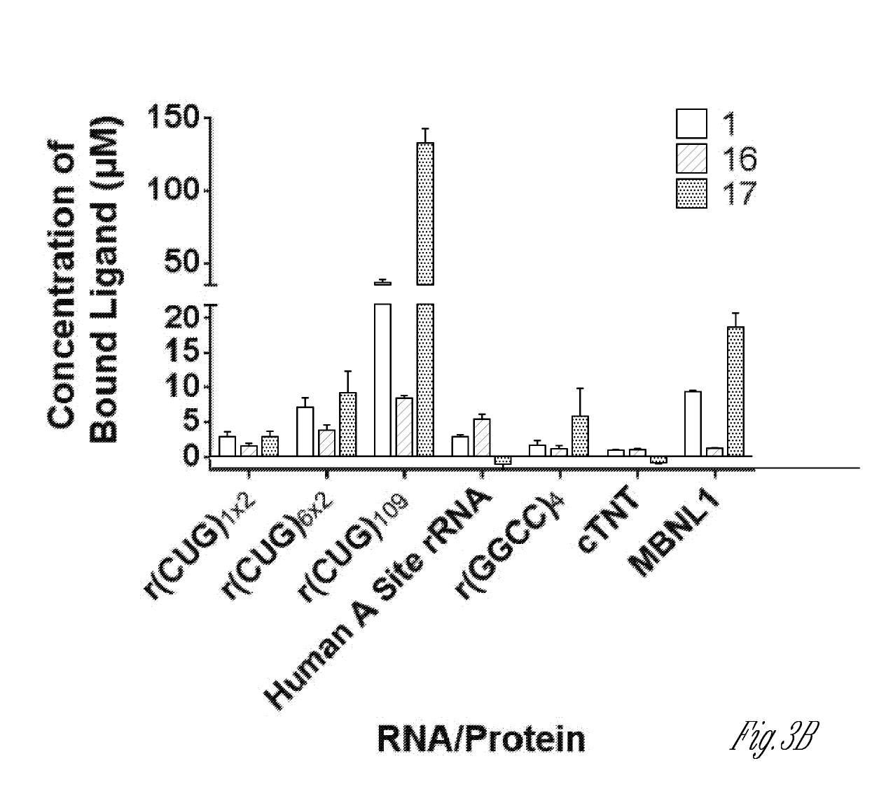

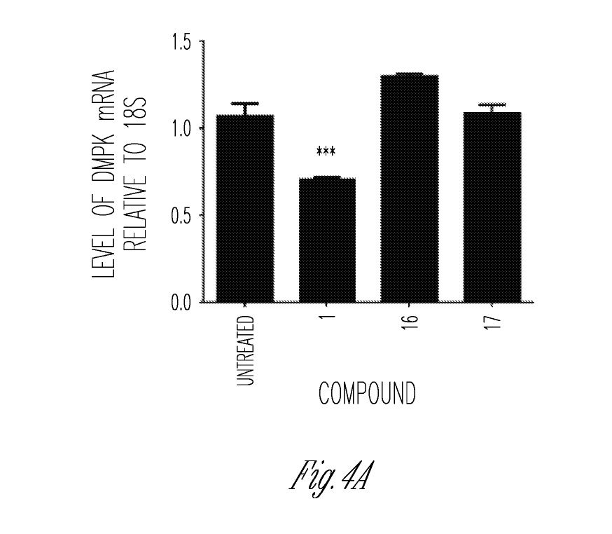

We selected compound 27 to produce more potent and selective r(CUG)^{exp}-targeting agent conforming to Formula (I). Compound 27 was selected as it had moderate activity in in vitro and cellular studies, was generally nontoxic and derivatives that could probe the effects of adding hydrogen bond donors and acceptors could be readily synthesized. To that end a bis-guanidino compound called 2GN8 was prepared for further evaluation (FIG. 5).

### In Vitro Evaluation of Potency and Selectivity of Lead-Optimized Compound 2GN8.

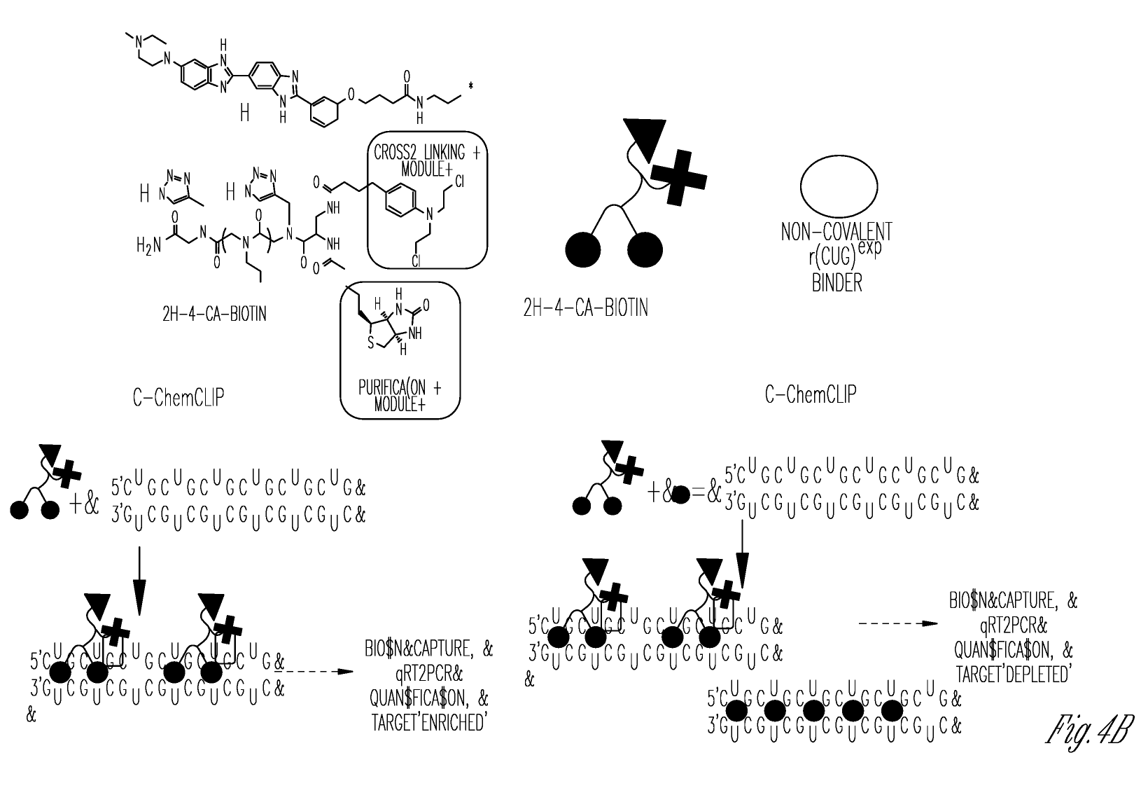

In vitro potency of 2GN8 was evaluated by TR-FRET assay and the IC₅₀ was determined to be 39±4 μM whereas the IC₅₀ of precursor compound 27 was found to be 531.8±82.8 μM. Thus simple modification resulted in roughly 14 fold enhancement in potency. Selectivity was assessed through competition dialysis experiments as described above. These experiments show that 2GN8 selectively binds r(CUG)^{exp} containing RNAs and has minimal interaction with other potential RNA motifs (FIG. 6).

### In Cellulo Evaluation of Selectivity of 2GN8.

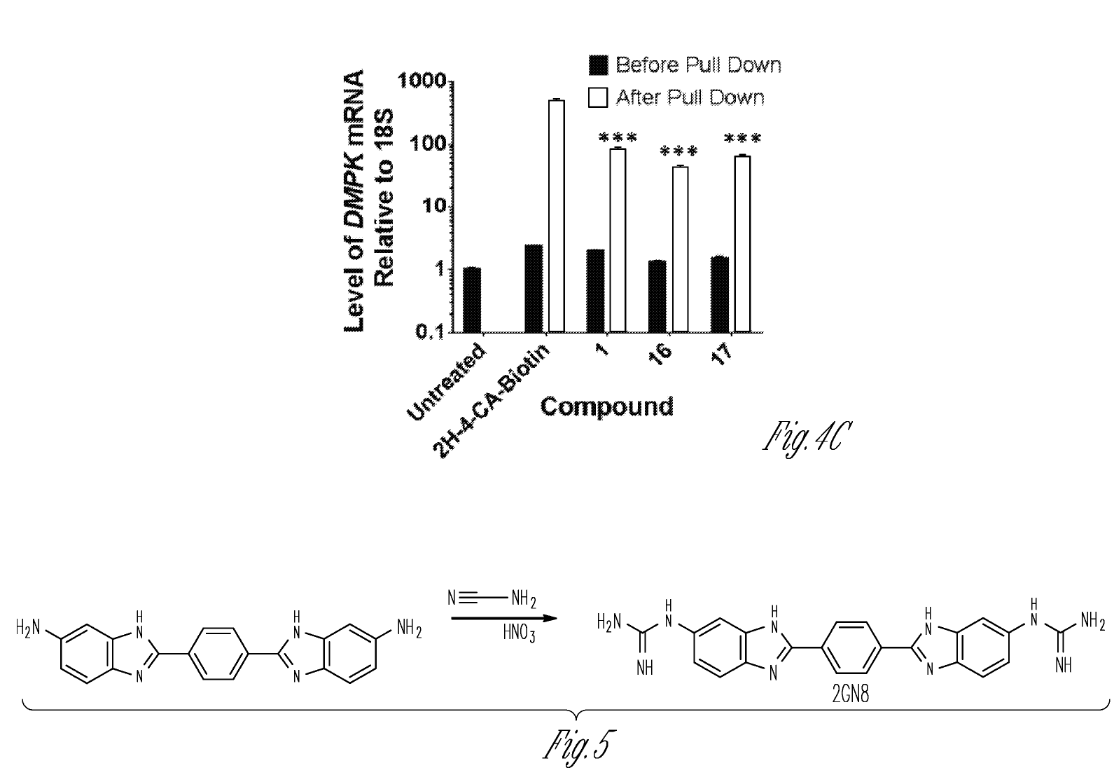

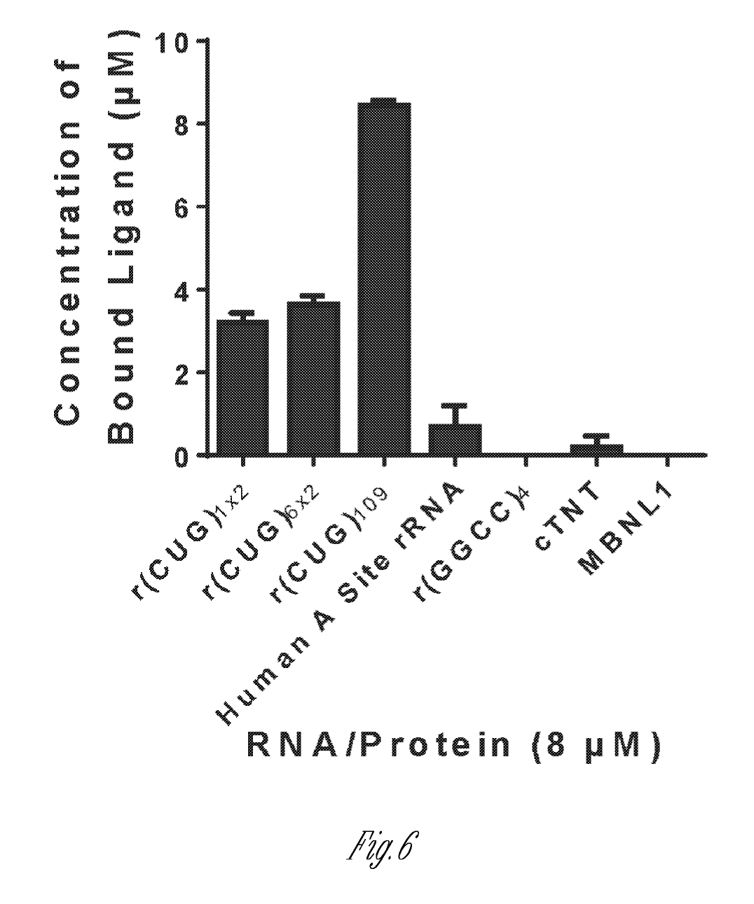

As mentioned above, in some previously reported studies, small molecules improve DM1-associated defects not by direct target engagement but rather by decreasing levels of r(CUG)^{exp} (DMPK) at the transcriptional level.⁽⁵¹⁾ Therefore, to validate the mechanism of action for 2GN8, we studied if it binds r(CUG)^{exp} in cellulo as well as the effect on the abundance of DMPK mnRNA. Evaluation of the effect of 2GN8 on the abundance of DMPK mRNA was assessed using patient derived fibroblasts with 500 CUG repeats. At bioactive concentrations, 2GN8 did not have any effect on the abundance of DMPK mRNA (FIG. 7).

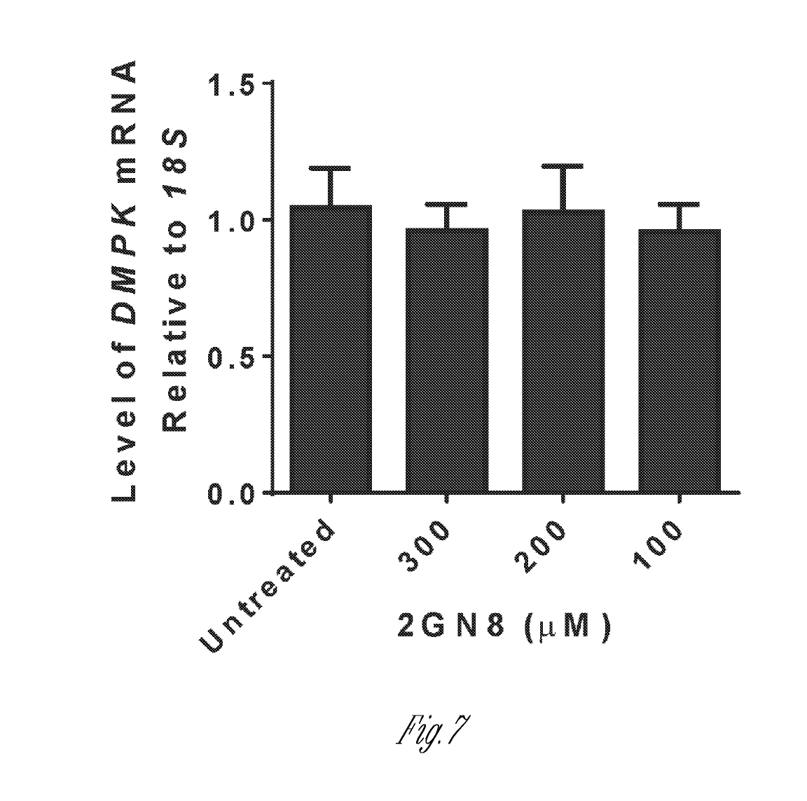

The biological target of 2GN8 was assessed using C-ChemCLIP as described above. In this particular application, we used C-ChemCLIP using a previously studied compound, 2H-4-CA-Biotin, that binds and cross-links to r(CUG)^{exp} in cellulo.⁽⁴⁹⁾ If 2GN8 also targets r(CUG)^{exp} in cellulo, then the amount of the RNA pulled down by 2H-4-CA-Biotin should decrease as a function of 2GN8 concentration. In the presence of 2GN8 the amount of r(CUG)^{exp} pulled down by 2H-4-CA-Biotin is reduced by half and indicates that 2GN8 binds r(CUG)^{exp} (FIG. 8).

### Evaluation of 2GN8 for Improvement of DM1 Translation Defects Using a Luciferase Reporter Assay.

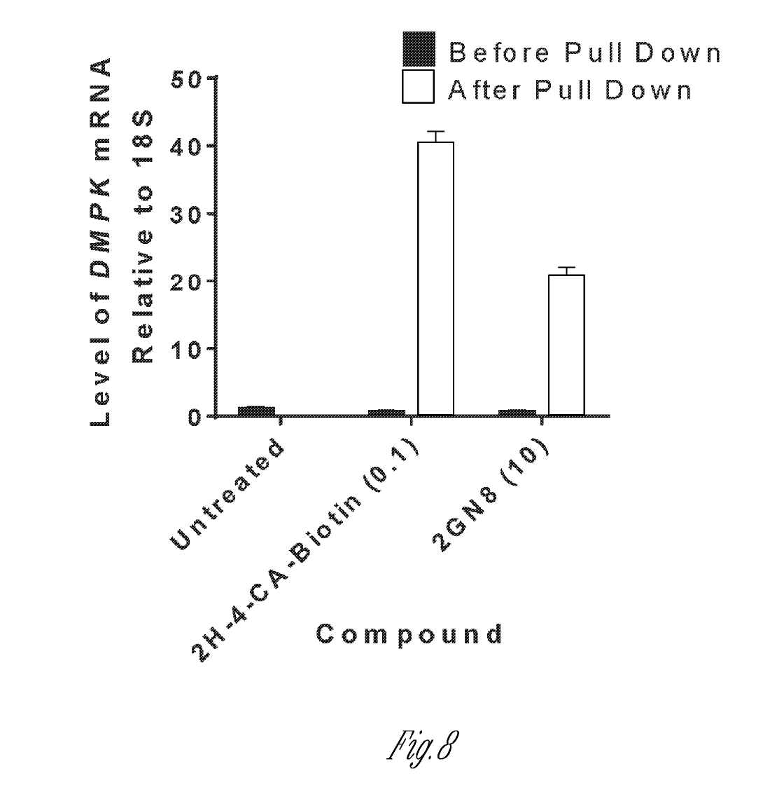

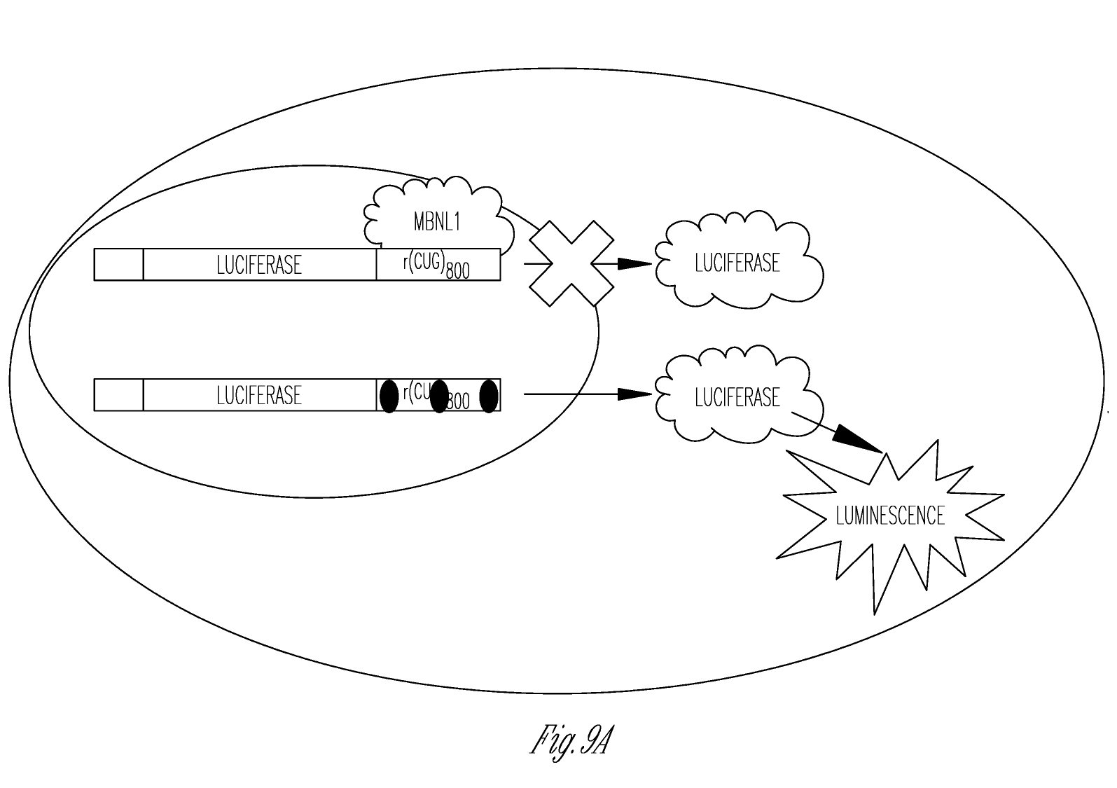

Exemplary Formula (I) compound 2GN8 was studied for improving translational defects associated with DM1 (due to poor nucleocytoplasmic transport of r(CUG)^{exp} containing transcripts).^{(30, 52, 53)} In particular, a cellular model system was employed in which the C2C12 cell line stably expresses r(CUG)₈₀₀ embedded in the 3′ UTR of firefly luciferase.⁽⁵⁴⁾ Similar to DMPK mRNA in DM1-affected cells, the expanded repeat impairs nucleocytoplasmic transport of luciferase mRNA and thus decreases luciferase expression. Compounds that bind r(CUG)^{exp} and disrupt the r(CUG)^{exp}-MBNL1 complex may stimulate cytoplasmic transport of the luciferase mRNA and thus translation of luciferase (FIG. 9A). A significant increase in luciferase activity (and hence improvement of the DM1-associated translational defect) is observed for 2GN8 (FIG. 9B). Importantly, 2GN8 does not affect luciferase activity of cells that stably express firefly luciferase mRNA without r(CUG)₈₀₀.

### Evaluation of 2GN8 for Improvement of DM1 Defects in Patient Derived Fibroblasts.

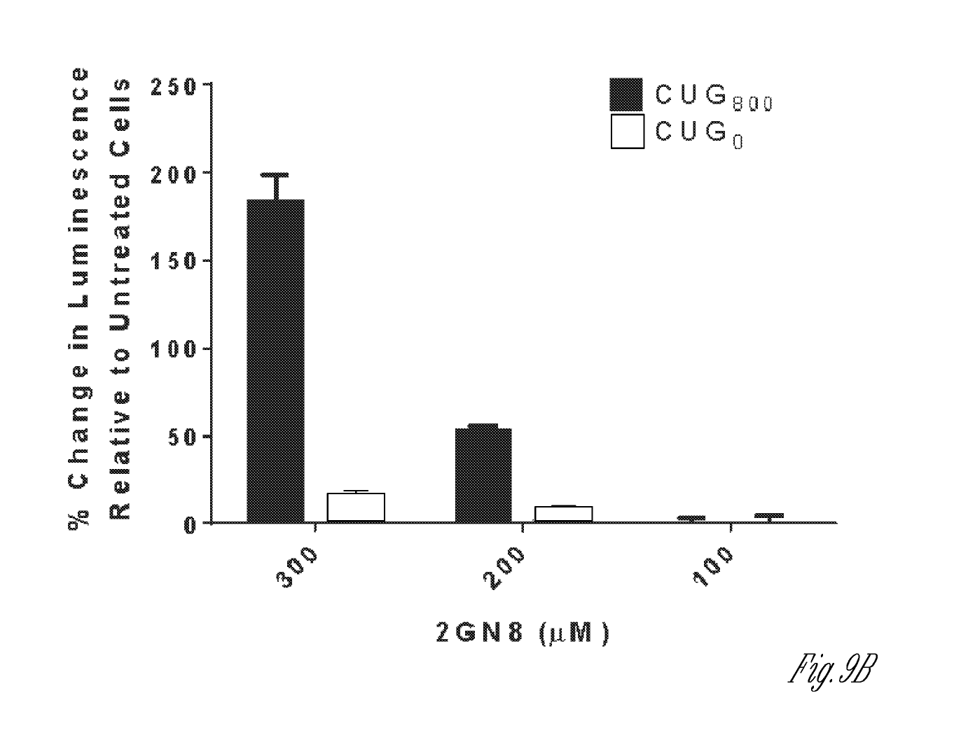

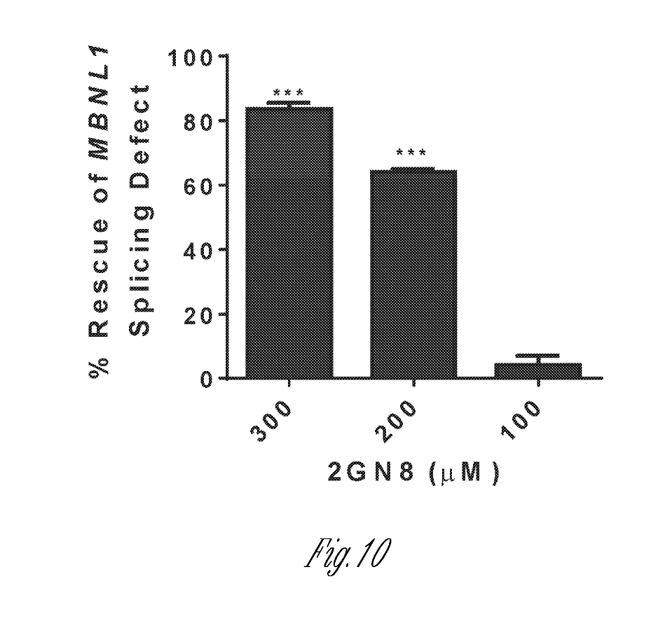

Bioactivity of 2GN8 was further assessed using DM1 patient derived fibroblasts containing 500 CUG repeats. First 2GN8 was evaluated for improvement of DM1 MBNL1 splicing defects. In DM1, mis-splicing of MBNL1 exon 5 results in an increase in inclusion to ˜30% compared to ˜15% inclusion in healthy tissue. At 300 and 200 μM, 2GN8 substantially improves MBNL1 pre-mRNA splicing defects (FIG. 10).

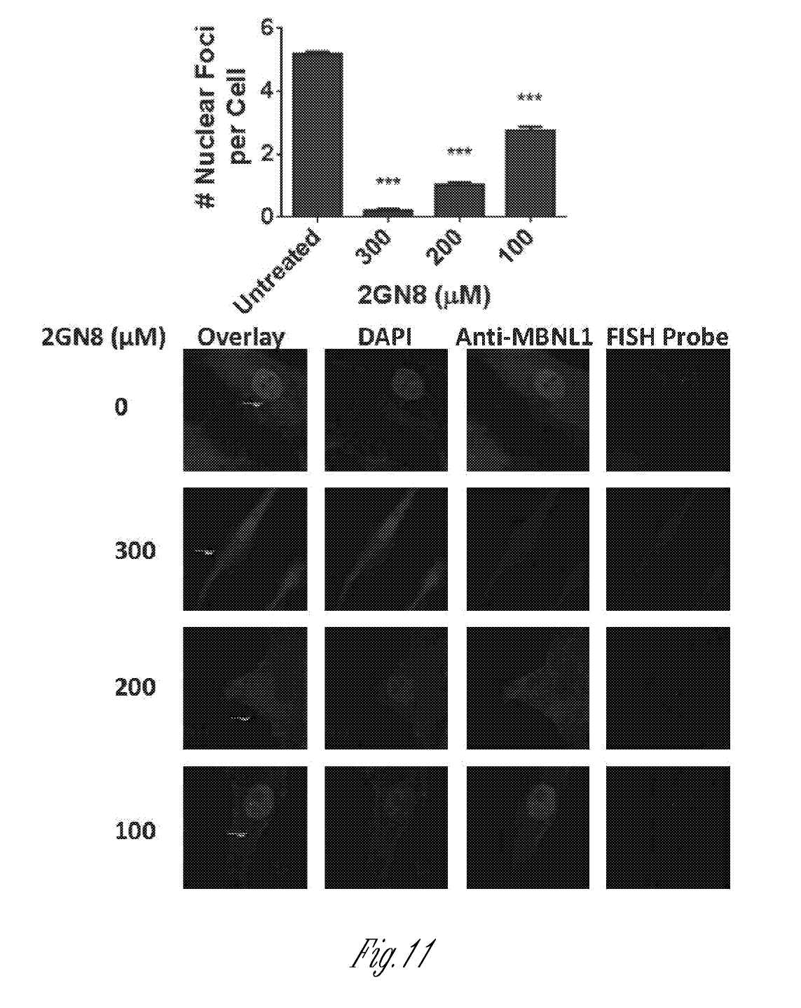

Bioactivity of 2GN8 was further assessed in DM1 patient derived fibroblasts for the disruption of nuclear foci. As observed in other microsatellite disorders, the binding of various proteins to r(CUG)^{exp} causes formation of nuclear foci.⁽⁵⁵⁾ Fluorescence in situ hybridization (FISH) with a dye-labeled oligonucleotide was used to determine if 2GN8 disrupts r(CUG)^{exp}-containing nuclear foci. In untreated cells, the average number of foci per cell is ˜5. Treatment with 2GN8 reduces the average number of foci per cell to less than 1 at 300 and 200 μM (FIG. 11).

### 2GN8 Improves Pre-mRNA Splicing and Myotonia in a Mouse Model of DM1.

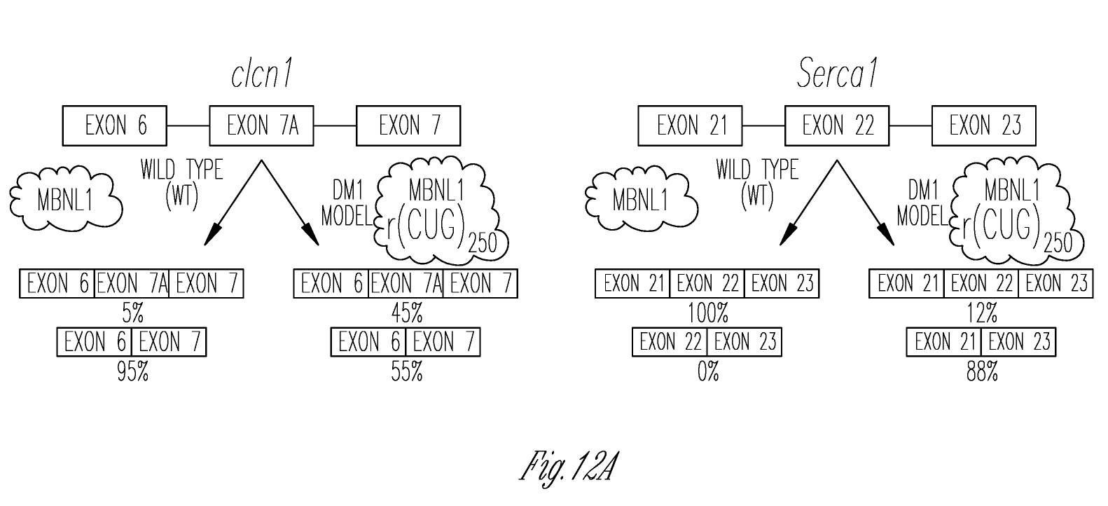

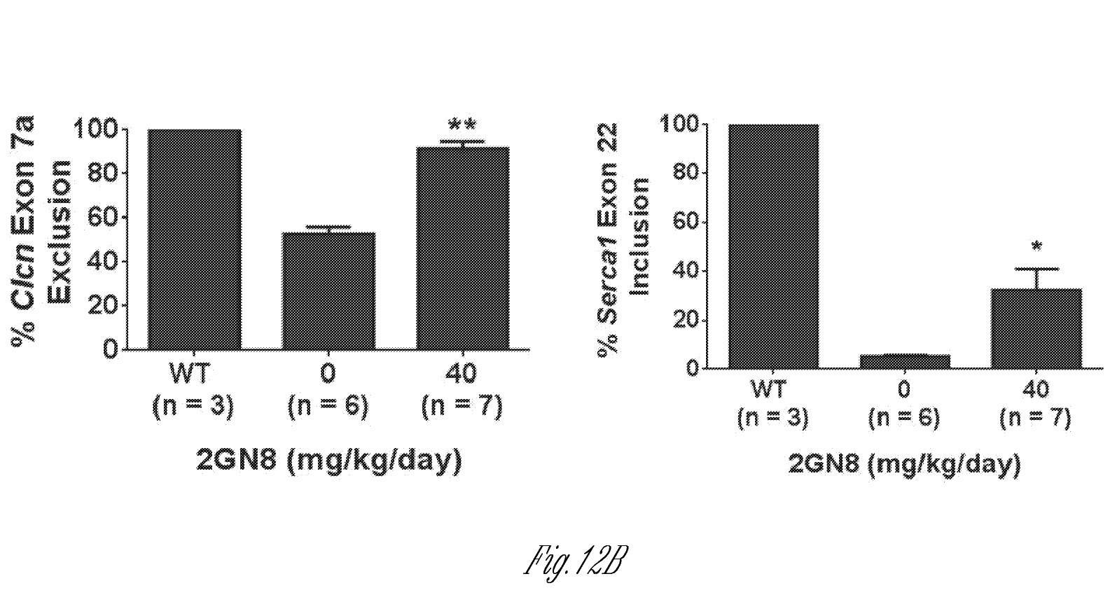

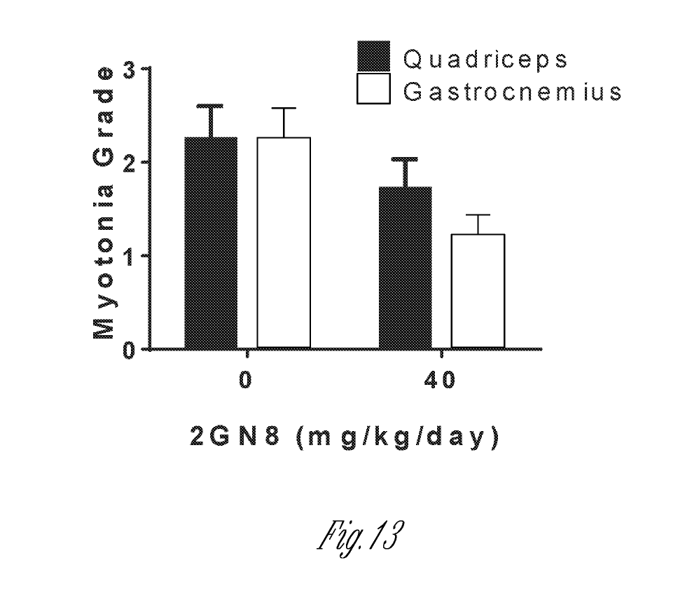

A mouse model of DM1 has been reported in which 250 rCUG repeats are expressed using an actin promoter (human skeletal actin long repeat, HSA^{LR}).²⁴ The presence of these repeats results in dysregulation of alternative splicing in the muscle-specific chloride ion channel (Clcn1) and the sarcoplasmic/endoplasmic reticulum calcium ATPase 1 (Serca1/Atp2a1) pre-mRNAs. Clcn1 exon 7a is excluded ˜100% in mRNA from normal adult mice: DM1 mice have an exclusion rate of ˜50% (FIG. 12A). After treating for 18 days with 40 mg/kg/day of 2GN8, pre-mRNA splicing defects in both Clcn1 and Serca1 were improved to nearly wild type levels (FIG. 12B). Also, myotonia grade was assessed in the quadriceps and gastrocnemius muscles and mice treated with 2GN8 had improved myotonia grade compared to untreated mice (FIG. 13).

### Compositions

In some embodiments, the invention also provides a pharmaceutical composition comprising one or more compounds according to Formula (I) or a pharmaceutically acceptable salt, solvate, stereoisomer, tautomer, or prodrug, in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents.

The inventive compositions can be administered orally, topically, parenterally, by inhalation or spray or rectally in dosage unit formulations. The term parenteral as used herein includes subcutaneous injections, intravenous, intramuscular, intrasternal injection or infusion techniques.

Suitable oral compositions in accordance with the invention include without limitation tablets, troches, lozenges, aqueous or oily suspensions, dispersible powders or granules, emulsion, hard or soft capsules, syrups or elixirs.

Encompassed within the scope of the invention are pharmaceutical compositions suitable for single unit dosages that comprise a compound of the invention its pharmaceutically acceptable stereoisomer, prodrug, salt, solvate, hydrate, or tautomer and a pharmaceutically acceptable carrier.

Inventive compositions suitable for oral use may be prepared according to any method known to the art for the manufacture of pharmaceutical compositions. For instance, liquid formulations of the inventive compounds contain one or more agents selected from the group consisting of sweetening agents, flavoring agents, coloring agents and preserving agents in order to provide pharmaceutically elegant and palatable preparations of the arginase inhibitor.

For tablet compositions, the active ingredient in admixture with non-toxic pharmaceutically acceptable excipients is used for the manufacture of tablets. Exemplary of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions the inventive compound is admixed with excipients suitable for maintaining a stable suspension. Examples of such excipients include without limitation are sodium carboxymethylcellulose, methylcellulose, hydropropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensaturatedion products of an alkylene oxide with fatty acids, for example polyoxyethylene stearate, or condensaturatedion products of ethylene oxide with long chain aliphatic alcohols, for example, heptadecaethyleneoxycetanol, or condensaturatedion products of ethylene oxide with partial esters derived from fatty acids and a hexitol such as polyoxyethylene sorbitol monooleate, or condensaturatedion products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensaturatedion products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of general Formula (I) may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

### Examples

The following examples are intended to illustrate certain embodiments of the invention, which is fully defined below by the claims. In addition, all publications cited herein are incorporated by reference as if fully set forth herein.

RNA-Focused Small Molecule Library Selection.

The TSRI small molecule library has approximately 1 million compounds available for screening efforts, comprising both diverse and focused sub-libraries. The drug discovery collection forms the largest part and consists of over 600,000 unique and drug-like compounds. It is constructed from commercial sources as well as past and current internal medicinal chemistry/drug discovery efforts. Compound management is via a customized chemical registration/search system that is built on ISIS/Host technology (Biovia, formerly Accelrys). A molecular similarity search, using the ISIS:Base client was performed to find compounds that were structurally similar to the reference compound. The degree of similarity is dependent on the percentage of searchable keys that the query compound has in common to a compound stored in the database. (ISIS sets approximately 960 searchable keys, defining structural features such as ring structure or heteroatom arrangement.) No compounds were found to be greater than 75% similar although a workable number (815) were found to be greater than 65%. Of these, 621 were available from the screening center from which the 320 compounds that are most chemically similar to the bis-benzimidazole lead (H1)³² were selected. This enabled the assay to be performed in a 384 well plate with space for controls. Chemical descriptors were then calculated in Pipeline Pilot (Biovia), including Lipinski's Rule of Five⁴⁰, PAINS⁴¹ and Lilly MedChem Rules⁴² via customized components that were created by the Scripps Molecular Screening Center. The final compounds were selected based on similarity score, availability from the Scripps Library (replenishment rules), and for purity and drug-likeness.

Small Molecules Evaluated in Cellular Assays.

Compounds 1, 7, 11, 16, and 17 were purchased from Asinex Ltd. Small molecules 2, 5, 6, 13, 15, 19, 21 and 24 were obtained from Scientific Exchange, Inc. Compounds 3, 9 and 26 were purchased from Chembridge. Compounds 8 and 25 were obtained from ChemDiv, Inc. Compounds 14, 23, and 27 were purchased from Sigma-Aldrich. Compound 20 was obtained from Ryan Scientific. Compound purity was assessed by analytical HPLC using a Waters Symmetry C18 5 μm 4.6×150 mm column. Compounds were analyzed using a gradient of 0-100% MeOH in 1H₂O with 0.1% TFA over 60 min. All compounds evaluated had ≥90% purity. The identities of compounds were confirmed by mass spectrometry using an α-hydroxycinnamic acid matrix and an Applied Biosystems MALDI ToF/ToF Analyzer 4800 Plus.

High Throughput Time-Resolved Fluorescence Resonance Energy Transfer (TR-FRET) screening.

The in vitro activity of library compounds was assessed by disruption of the r(CUG)₁₂-MBNL1 complex using a previously reported TR-FRET assay.^{(32, 46)} Biotinylated RNA was folded at 60° C. in 1× Folding Buffer (20 mM HEPES, pH 7.5, 100 M KCl, and 10 mM NaCl) and slowly cooled to room temperature. The buffer was then adjusted to 1× Assay Buffer (1× Folding Buffer supplemented with 2 mM MgCl₂, 2 mM CaCl₂, 5 mM DTT, 0.1% BSA, 0.05% Tween-20) and MBNL1-His₆ was added. The final concentrations of RNA and MBNL1-His₆ were 80 nM and 62 nM, respectively. After incubation at room temperature for 15 min, 8 μL aliquots were then dispensed into each well of low volume, 384-well plates using a flying reagent dispenser. A 400 nL aliquot of small molecule stock solution (2.5 mM) was added using a Beckman Coulter Biomek NX^{P} Laboratory Automation Workstation, affording a final small molecule concentration of ˜100 μM. The samples were allowed to equilibrate at room temperature for 15 min, after which 1 μL of antibody solution (1:1 mixture of 8.8 ng/μL Anti-His₆-Tb and 800 nM streptavidin XL-665) was dispensed into the wells. Control wells for maximum TR-FRET (100% complex formation) contained 9 μL of 1× Assay Buffer and 1 μL water. Controls for minimum TR-FREFT (no complex formation) contained 9 μL of 1× Assay Buffer, 1 μL water, and no RNA or protein. Plates were incubated at room temperature for 1 h, and then TR-FRET was measured using a Molecular Devices SpectraMax M5 plate reader using an excitation wavelength of 345 nm and a 420 nm cutoff. Fluorescence was measured at 545 and 665 nm. Library compounds were screened for potential intrinsic fluorescence under screening conditions. No library compounds were fluorescent at 665 nm and two non-hit compounds have low fluorescence at 545 nm.

To calculate the percent inhibition of complex formation, the ratio of fluorescence intensity at 545 nm and 665 nm in the presence of compound was compared to the ratio in the absence of small molecule (100% r(CUG)₁₂-MBNL1 complex formation) and in the absence of RNA and protein (no complex formation). Hit compounds had a percent inhibition ≥3 times the standard deviation of the percent inhibition of the entire plate.

Improvement of DM1-Associated Splicing Defects in a DM1 Cell Culture Model Using RT-PCR.

In order to determine bioactivity of hit compounds as assessed by improvement of DM1-associated alternative pre-mRNA splicing defects, a previously reported method was utilized.^{(25, 63)} Briefly, HeLa cells were grown as monolayers in 96-well plates in growth medium (1×DMEM, 10% FBS, and 1× Glutamax (Invitrogen)). Once the cells reached 80% confluence, they were transfected with 200 ng of total plasmid using Lipofectamine 2000 (Invitrogen) according to the manufacturer's standard protocol. Equal amounts of plasmid expressing a DM1 mini-gene with 960 CTG repeats⁽²⁵⁾ and mini-gene that reports on cTNT alternative splicing (regulated by MBNL1)⁽²⁵⁾ were used. After 5 h, the transfection cocktail was removed and replaced with growth medium containing the compound of interest. After 20-24 h the cells were lysed, and total RNA was harvested using a Zymo Quick RNA miniprep kit. An on-column DNA digestion was completed per the manufacturer's recommended protocol. A sample of RNA was subjected to RT-PCR as previously described⁶⁰ using ˜150 ng of total RNA.

The RT-PCR primers for the cTNT mini-gene were 5′-GTTCACAACCATCTAAAGCAAGATG (forward) and 5′-GTTGCATGGCTGGTGCAGG (reverse). The forward primer was radiolabeled with γ-³²P ATP using T4 polynucleotide kinase. RT-PCR products were separated using a denaturing 5% polyacrylamide gel run at 50 W for 2 h in 1×TBE buffer. Gels were imaged using a Molecular Dynamics Typhoon 9410 variable mode imager.

Competition Dialysis.

Competition dialysis was performed using Pierce Slide-A-Lyzer MINI dialyzer units with a 3,500 Da molecular weight cutoff. The RNAs evaluated were folded by heating an 8 μM solution of RNA in 199 μL 1× Dialysis Buffer (8 mM NaH₂PO₄, pH 7.2, 185 mM NaCl and 1 mM EDTA) at 65° C. for 5 min followed by slowly cooling to room temperature on the bench top. Then, 1 μL of 1 mM small molecule was added (5 μM final concentration), and the solution was transferred to the dialyzer units (100 μL samples in duplicate). The dialyzer units were then placed in 150 mL of 5 μM small molecule in 1× Dialysis Buffer and stirred at 200 rpm at 4° C. for 48 h. Following equilibration, 90 μL of each sample was transferred to a microcentrifuge tube and treated with 10 μL of 10% SDS to dissociate the ligand. The total ligand concentration (Cₜ) within each dialysis unit was quantified spectrophotometrically using the appropriate absorbance wavelength and extinction coefficient for each compound. The free ligand concentration (C_{f}) was determined from a sample of the dialysate solution, which did not vary significantly from the initial concentration. The bound ligand concentration (C_{b}) was then determined using equation 1:

C_{b}=Cₜ−C_{f} (eq. 1)

where C_{b}, Cₜ, and C_{f} are concentrations of bound, total, and free ligand, respectively.

Target Pull-Down by C-ChemnCLIP and Analysis by qRT-PCR.

HeLa cells were grown as monolayers to 80% confluence in 100 mm³ plates in growth medium. Cells were transfected with 10 μg of plasmid expressing the DM1 mini-gene using Lipofectamine 2000 (Invitrogen) according to the manufacturer's standard protocol. After 5 h, the transfection cocktail was removed and replaced with growth medium containing 100 nM 2H-4-CA-Biotin and 10 μM bioactive small molecule. After 20-24 h, the cells were lysed and total RNA was harvested using Trizol reagent (Ambion) according to the manufacturer's protocol. Approximately 30 μg of total RNA was used for pull-down using 15 nmoles of streptavidin-agarose beads (Sigma-Aldrich; 15 μg/mL biotin loading) as previously described.⁽⁴⁹⁾ The lysate was incubated with the beads for 1 h at room temperature with shaking at 500 rpm. The solution was removed, and the beads were washed with 500 μL aliquots of 1×PBS. Bound material was released from the beads by heating at 95° C. for 5 min in 30 μL of 95% formamide containing 10 mM EDTA, pH 8.2.

Reverse transcription reactions were carried out using qScript cDNA synthesis kit by adding ˜60 ng of either total RNA or captured RNA according to the manufacturer's protocol. Then, 30% of each cDNA sample was used for quantitative real time PCR (qPCR) analysis for each primer set. qPCR was performed on a 7900HT Fast Real-Time PCR System (Applied Biosystems) using Power SYBR Master Mix (Life Technologies). The PCR primers for the DM1/DMPK mini-gene mRNA were 5′-CGTGCAAGCGCCCAG (forward) and 5′-CTCCACCAACTTACTGTTTCATTCT (reverse). The PCR primers for 18S ribosomal RNA were 5′-GTAACCCGTTGAACCCCATT (forward) and 5′-CCATCCAATCGGTAGTAGCG (reverse).

Analysis of the Effects of Small Molecules on DMPK mRNA Abundance by qRT-PCR.

HeLa cells were grown as monolayers to 80% confluence in 6-well plates in growth medium. Cells were transfected with 2.5 μg/well of total plasmid using Lipofectamine 2000 (Invitrogen) according to the manufacturers standard protocol. Equal amounts of plasmids expressing the DM1/DMPK and cTNT mini-genes were used. After 5 h, the transfection cocktail was removed and replaced with growth medium containing 100 μM small molecule. After 20-24 h, total RNA was isolated as described above. Reverse transcription reactions were carried out using a qScript cDNA synthesis kit by adding ˜200 ng of total RNA according to the manufacturer's protocol. The qRT-PCR analyses were carried out as described above.

Toxicity Analysis of Bioactive Compounds.

The toxicities of 1, 16, and 17 were assessed using our model system for DM1 pre-mRNA splicing defects as described above. After treatment with compound for 24 h, the cells were washed with 1×DPBS and treated with 100 μL of a 10% solution of WST-1 reagent (Roche) in growth medium. Cells were incubated with this solution for 30 min at 37° C. and then 60 μL aliquots were transferred to a 96-well plate. The absorbance at 450 nm and 690 nm was measured using a Molecular Devices SpectraMax M5 plate reader.

Synthesis of 2GN8.

2,2′-p-Phenylene-bis(5-aminobenzimidazole) was synthesized as previously described(64) and 150 mg (0.44 mmol) was suspended in water (1.5 mL) and ethanol (0.5 mL). Cyanamide (730 mg, 17.6 mmol) was added followed by 6 drops of concentrated nitric acid. This was reacted in a Biotage Initiator+ SP Wave microwave at 130° C. for 6 h. Then the reaction mixture was diluted with water and purified by preparative reverse phase HPLC using a Waters 1525 Binary HPLC pump equipped with a Waters 2487 dual absorbance detector system and a Waters Sunfire C18 OBD 5 μm 19×150 mm column. Absorbance was monitored at 345 and 220 nm. A gradient of 0-100% MeOH in H₂O with 0.1% TFA over 60 min was used for compound purification. Isolated 50 mg of product (27%, yellow solid). H¹NMR (400 mHz, CD₃OD): δ 8.36 (s, 4H), 7.79 (d, 2H, J=9), 7.66 (d, 2H, J=2), 7.33 (dd, 2H, J=2, 9).

Time-Resolved Fluorescence Resonance Energy Transfer (TR-FRET) Assay.

Initial in vitro activity of 2GN8 was assessed by disruption of the r(CUG)^{exp}-MBNL1 complex using a previously reported TR-FRET assay.(32, 46) The ratio of fluorescence intensity of 545 and 665 nm as compared to the ratios in the absence of small molecule and in the absence of RNA were used to calculate percent inhibition. The resulting curves were fit to the following equation to determine IC₅₀ values:

$$y = B + \frac{A - B}{1 + {( \frac{IC 50}{x} )}^{hillslope}}$$

where y is ratio of fluorescence intensities at 545 nm and 665 nm (F545/F665), x is the concentration of small molecule, B is F545/F665 value at max FRET effect (solution has RNA and protein but no small molecule added); A is F545/F665 value at rain FRET effect (solution has antibodies but no RNA, protein, or small molecule); and the IC₅₀ is the concentration of small molecule where half of the protein is displaced by small molecule. The IC₅₀ of 2GN8 was determined to be 39±4 μM.

Evaluation of DM1 Splicing Defects in Patient Derived Fibroblasts.

Bioactivity of 2GN8 was assessed by using DM1 patient derived fibroblasts containing 500 CTG repeats (GM03987) and healthy fibroblasts (GM07492). Cells were grown as monolayers in 6 well plates in growth medium (1×EMEM (Lonza), 10% FBS, 1× glutagro (Corning), 1×MEM non-essential amino acids (Corning) and 1× antibiotic/antimycotic (Corning)). Once cells were ˜80% confluent, they were treated with growth medium containing 2GN8 (300, 200 and 100 μM). After 48 h the cells were lysed and the total RNA was harvested using a Zymo Quick RNA miniprep kit. An on-column DNA digestion was completed per the manufacturer's recommended protocol. Approximately 100 ng of total RNA was reverse transcribed at 50° C. using 100 units of SuperScript III reverse transcriptase (Life Technologies). Then 20% of the RT reaction was subjected to PCR using GoTaq DNA polymerase. RT-PCR products were observed after 25 cycles of 95° C. for 30 s, 58° C. for 30 s, 72° C. for 1 min and a final extension at 72° C. for 1 min. The products were separated on an 2% agarose gel ran at 100 V for 1 h in 1×TAE buffer. The products were visualized by staining with ethidium bromide and scanned using a Bio-Rad Gel Doc XR+ imaging system. The RT-PCR primers for the MBNL1 were 5′GCTGCCCAATACCAGGTCAAC (forward) and 5′TGGTGGGAGAAATGCTGTATG (reverse).

Evaluation of Target Selectivity of 2GN8 Using Competition Dialysis.

Competition dialysis was performed using Pierce Slide-A-Lyzer MINI dialyzer units with a 3,500 Da molecular weight cutoff. The RNAs evaluated were folded by heating an 8 μM solution of RNA in 199 μL 1× Dialysis Buffer (8 mM NaH₂PO₄, pH 7.2, 185 mM NaCl and 1 mM EDTA) at 65° C. for 5 min followed by slowly cooling to room temperature on the bench top. Then, 1 μL of 1 mM small molecule was added (5 μM final concentration), and the solution was transferred to the dialyzer units (100 μL samples in duplicate). The dialyzer units were then placed in 150 mL of 5 μM small molecule in 1× Dialysis Buffer and stirred at 200 rpm at 4° C. for 48 h. Following equilibration, 90 μL of each sample was transferred to a microcentrifuge tube and treated with 10 μL of 10% SDS to dissociate the ligand. The total ligand concentration (Cₜ) within each dialysis unit was quantified spectrophotometrically using the appropriate absorbance wavelength and extinction coefficient for each compound. The free ligand concentration (C_{f}) was determined from a sample of the dialysate solution, which did not vary significantly from the initial concentration. The bound ligand concentration (C_{b}) was then determined using equation 1:

C_{b}=Cₜ−C_{f} (eq. 1)

where C_{b}, Cₜ, and C_{f} are concentrations of bound, total, and free ligand, respectively.

Target Pull-Down by C-ChemCLIP and Analysis by qRT-PCR.

HeLa cells were grown as monolayers to 80% confluence in 75 mm² dishes in growth medium (1× DMEM, 10% FBS, and 1× Glutamax (Invitrogen)). Cells were transfected with 10 μg of plasmid expressing the DM1 mini-gene using Lipofectamine 2000 (Invitrogen) according to the manufacturer's standard protocol. After 5 h, the transfection cocktail was removed and replaced with growth medium containing 100 nM 2H-4-CA-Biotin and 10 μM 2GN8. After 24 h, the cells were lysed and total RNA was harvested using Trizol reagent (Ambion) according to the manufacturer's protocol. Approximately 30 μg of total RNA was used for pull-down using 15 nmoles of streptavidin-agarose beads (Sigma-Aldrich: 15 μg/mL biotin loading) as previously described.⁽⁴⁹⁾ The lysate was incubated with the beads for 1 h at room temperature with shaking at 500 rpm. The solution was removed, and the beads were washed with 500 μL aliquots of 1×PBS. Bound material was released from the beads by heating at 95° C. for 5 min in 30 μL of 95% formamide containing 10 mM EDTA, pH 8.2.

Reverse transcription reactions were carried out using qScript cDNA synthesis kit by adding 150 ng of either total RNA or captured RNA according to the manufacturer's protocol. Then, 30% of each cDNA sample was used for quantitative real time PCR (qPCR) analysis for each primer set. qPCR was performed on a 7900HT Fast Real-Time PCR System (Applied Biosystems) using Power SYBR Master Mix (Life Technologies). The PCR primers for the DM1/DMPK mini-gene mRNA were 5′-CGTGCAAGCGCCCAG (forward) and 5′-CTCCACCAACTTACTGTTTCATTCT (reverse). The PCR primers for 18S ribosomal RNA were 5′-GTAACCCGTTGAACCCCATT (forward) and 5′-CCATCCAATCGGTTAGCG (reverse).

Analysis of the Effects of 2GN8 on DMPK mRNA Abundance by qRT-PCR.

The effect of 2GN8 on the abundance of DMPK mRNA was assessed using DM1 patient derived fibroblasts containing 500 CTG repeats (GM03987). Cells were grown as monolayers in 6 well plates in growth medium (1×EMEM (Lonza), 10% FBS, 1× glutagro (Corning), 1×MEM non-essential amino acids (Corning) and 1× antibiotic/antimycotic (Corning)). Once cells were ˜80% confluent, they were treated with growth medium containing the 2GN8 (300, 200 and 100 μM). After 48 h the cells were lysed and the total RNA was harvested using Trizol reagent (Life Technologies). Approximately 100 ng of RNA was used for RT qScript cDNA synthesis kit (Quanta BioSciences). 30% of the RT reaction was used for real time PCR (qPCR) with SYBR green master mix (Life Technologies) performed on a 7900HT Fast Real-Time PCR System (Applied Biosystems). The PCR primers for DMPK mRNA were 5′-CGTGCAAGCGCCCAG (forward) and 5′-CTCCACCAACTTACTGTTTCATCCT (reverse). The PCR primers for 18S ribosomal RNA were 5′-GTAACCCGTTGAACCCCATT (forward) and 5′-CCATCCAATCGGTAGTAGCG (reverse).

Evaluation of Nuclear Foci Using Fluorescence In Situ Hybridization (FISH).

FISH was utilized to determine the effects of 2GN8 on the formation and disruption of nuclear foci. DM1 patient derived fibroblasts containing 500 CTG repeats (GM03987) were grown to ˜80% confluence in a Mat-Tek 96-well glass bottom plate in growth medium. Cells were treated with 2GN8 (300, 200 and 100 μM) for 48 h in growth medium followed by FISH as previously described(63) using 1 ng/μL DY547-2′OMe-(CAG)₆. Immunostaining of MBNL1 was completed as previously described(65) using the MB1a antibody (diluted 1:4), which was generously supplied by Prof. Glenn E. Morris (Wolfson Centre for Inherited Neuromuscular Disease(65). This was fluorescently labeled using a 1:200 dilution of goat anti-mouse IgG DyLight 488 conjugate. Untreated controls were stained using a 1 μg/μL solution of DAPI in 1×DPBS. Cells were imaged in 1×DPBS using an Olympus FluoView 1000 confocal microscope at 100× magnification.

Evaluation of Translational Defects Using a Luciferase Reporter Assay.

C2C12 cell lines expressing 800 or 0 CTG repeats in the 3′ UTR of luciferase were grown as monolayers in 96-well plates in growth medium (1×DMEM, 10% FBS, 1× glutagro, (Corning) and 1× antibiotic/antimycotic (Corning))(66). Once the cells were 80% confluent, 2GN8 (300, 200 and 100 μM) was added in 100 μL of growth medium. Cells were treated for 48 h and then the cell count was normalized using WST-1 reagent (Roche). Then cells were washed with 1×DPBS and lysed by treating with 50 μL of PPBT lysis buffer at room temperature for 10 minutes. Then 50 μL of luciferase substrate was added and luminescence was measured.

Evaluation of Splicing Defects Using a DM1 Mouse Model.

All experimental procedures, mouse handling, and husbandry were completed in accordance with the Association for Assessment and Accreditation of Laboratory Animal Care. A mouse model for DM1(67). HSALR in line 20b, was used. HSALR mice express human skeletal actin RNA with 250 CUG repeats in the 3′ UTR. Age- and gender-matched HSALR mice were injected intraperitoneally with either 40 mg/kg 2GN8 in water for treatment and 0.9% NaCl for control once per day for 18 days. Mice were sacrificed one day after the last injection, and the vastus muscle was obtained. RNA was extracted from the vastus tissue, and cDNA was synthesized as previously described.

### CITED PUBLICATIONS

1. St. Laurent, G., Faghihi, M. A., and Wahlestedt, C. (2009) Non-coding RNA transcripts: Sensors of neuronal stress, modulators of synaptic plasticity, and agents of change in the onset of Alzheimer's disease, Neuroscience letters 466, 81-88.
2. Qureshi, I. A., and Mehler, M. F. (2013) Long Non-coding RNAs: Novel Targets for Nervous System Disease Diagnosis and Therapy, Neurotherapeutics 10, 632-646.
3. Calin, G. A., and Croce, C. M. (0000) MicroRNAs and chromosomal abnormalities in cancer cells, Oncogene 25, 6202-6210.
4. Blount, K. F., and Breaker, R. R. (2006) Riboswitches as antibacterial drug targets, Nat Biotechnol 24, 1558-1564.
5. Blount, K. F., Wang, J. X., Lim, J., Sudarsan, N., and Breaker, R. R. (2007) Antibacterial lysine analogs that target lysine riboswitches, Nat Chem Biol 3, 44-49.
6. Alvarez-Salas, L. M. (2008) Nucleic acids as therapeutic agents, Curr Top Med Chem 8, 1379-1404.
7. Wheeler, T. M., Sobczak, K., Lueck, J. D., Osborne, R. J., Lin, X., Dirksen, R. T., and Thornton, C. A. (2009) Reversal of RNA Dominance by Displacement of Protein Sequestered on Triplet Repeat RNA, Science 325, 336-339.
8. Wheeler, T. M., Lueck, J. D., Swanson, M. S., Dirksen, R. T., and Thornton, C. A. (2007) Correction of CIC-1 splicing eliminates chloride channelopathy and myotonia in mouse models of myotonic dystrophy, The Journal of Clinical Investigation 117, 3952-3957.
9. Mulders, S. A. M., van den Broek, W. J. A. A., Wheeler, T. M., Croes, H. J. E., van Kuik-Romeijn, P., de Kimpe, S. J., Furling, D., Platenburg, G. J., Gourdon, G., Thornton, C. A., Wieringa, B., and Wansink, D. G. (2009) Triplet-repeat oligonucleotide-mediated reversal of RNA toxicity in myotonic dystrophy, Proceedings of the National Academy of Sciences of the United States of America 106, 13915-13920.
10. Sobczak, K., Wheeler, T. M., Wang, W., and Thornton, C. A. (2013) RNA Interference Targeting CUG Repeats in a Mouse Model of Myotonic Dystrophy, Mol Ther 21, 380-387.
11. Velagapudi, S. P., Gallo, S. M., and Disney, M. D. (2014) Sequence-based design of bioactive small molecules that target precursor microRNAs, Nat Chem Biol 10, 291-297.
12. Sudarsan, N., Wickiser, J. K., Nakamura, S., Ebert, M. S., and Breaker, R. R. (2003) An mRNA structure in bacteria that controls gene expression by binding lysine, Genes & Development 17, 2688-2697.
13. Kubodera, T., Watanabe, M., Yoshiuchi, K., Yamashita, N., Nishimura, A., Nakai, S., Gomi, K., and Hanamoto, H. (2003) Thiamine-regulated gene expression of Aspergillus oryzae thiA requires splicing of the intron containing a riboswitch-like domain in the 5′-UTR, FEBS Letters 555, 516-520.
14. Sudarsan, N., Cohen-Chalaamish, S., Nakamura, S., Emilsson, G. M., and Breaker, R. R. (2005) Thiamine Pyrophosphate Riboswitches Are Targets for the Antimicrobial Compound Pyrithiamine, Chem. Biol. 12, 1325-1335.
15. Fourmy, D., Recht, M. I., Blanchard, S. C., and Puglisi, J. D. (1996) Structure of the Å Site of Escherichia coli 16S Ribosomal RNA Complexée! with an Aminoglycoside Antibiotic, Science 274, 1367.
16. Hermann, T., and Westhof, E. (1998) Aminoglycoside binding to the hammerhead ribozyme: a general model for the interaction of cationic antibiotics with RNA, Journal of Molecular Biology 276, 903-912.
17. Yoshizawa, S., Fournmy, D., and Puglisi, J. D. (1998) Structural origins of gentamicin antibiotic action, The EMBO Journal 17, 6437-6448.
18. Niu, Y., Jones, A. J., Wu, H, Varani, G., and Cai, J. (2011) γ-AApeptides bind to RNA by mimicking RNA-binding proteins, Organic & biomolecular chemistry 9, 10.1039/c1031 ob05738c.
19. Davidson, A., Leeper, T. C., Athanassiou, Z., Patora-Komisarska, K., Karn, J., Robinson, J. A., and Varani, G. (2009) Simultaneous recognition of HIV-1 TAR RNA bulge and loop sequences by cyclic peptide mimics of Tat protein, Proceedings of the National Academy of Sciences 106, 11931-11936.
20. Kwon, Y., Arndt, H.-D., Mao, Q., Choi, Y., Kawazoe, Y., Dervan, P. B., and Uesugi, M. (2004) Small Molecule Transcription Factor Mimic, Journal of the American Chemical Society 126, 15940-15941.
21. Lind, K. E., Du, Z. Fujinaga, K., Peterlin, B. M., and James, T. L. (2002) Structure-based computational database screening, in vitro assay, and NMR assessment of compounds that target TAR RNA, Chem Biol 9, 185-193.
22. Brook, J. D., McCurrach, M. E., Harley, H. G., Buckler, A. J., Church, D., Aburatani, H., Hunter, K., Stanton, V. P., Thirion, J. P., Hudson, T., Sohn, R., Zemelman, B., Snell, R. G., Rundle, S. A., Crow, S., Davies, J., Shelbourne, P., Buxton, J., Jones, C., Juvonen, V., Johnson, K., Harper, P. S., Shaw, D. J., and Housman, D. E. (1992) Molecular basis of myotonic dystrophy: expansion of a trinucleotide (CTG) repeat at the 3′ end of a transcript encoding a protein kinase family member, Cell 68, 799-808.
23. Tsilfidis, C., MacKenzie, A. E., Mettler, G., Barcelo, J., and Korneluk, R. G. (1992) Correlation between CTG trinucleotide repeat length and frequency of severe congenital myotonic dystrophy, Nat Genet 1, 192-195.
24. Mankodi, A., Takahashi, M. P., Jiang, H., Beck, C. L., Bowers, W. J., Moxley, R. T., Cannon, S. C., and Thornton, C. A. (2002) Expanded CUG repeats trigger aberrant splicing of C1C-1 chloride channel pre-mRNA and hyperexcitabiity of skeletal muscle in myotonic dystrophy, Mol Cell 10, 35-44.
25. Philips, A. V., Timchenko, L. T., and Cooper, T. A. (1998) Disruption of splicing regulated by a CUG-binding protein in myotonic dystrophy, Science 280, 737-741.
26. Taneja, K. L., McCurrach, M., Schalling, M., Housman, D., and Singer, R. H. (1995) Foci of trinucleotide repeat transcripts in nuclei of myotonic dystrophy cells and tissues, J Cell Biol 128, 995-1002.
27. Miller, J. W., Urbinati, C. R., Teng-Umnuay, P., Stenberg, M. G., Byrne, B. J., Thornton, C. A, and Swanson, M. S. (2000) Recruitment of human muscleblind proteins to (CUIG)(n) expansions associated with myotonic dystrophy, Embo J 19, 4439-4448.
28. Charlet, B. N., Savkur, R. S., Singh, G., Philips, A. V., Grice, E. A., and Cooper, T. A. (2002) Loss of the muscle-specific chloride channel in type 1 myotonic dystrophy due to misregulated alternative splicing, Mol Cell 10, 45-53.
29. Fardaei, M., Larkin, K., Brook, J. D., and Hamshere, M. G. (2001) In vivo co-localisation of MBNL protein with DMPK expanded-repeat transcripts, Nucleic Acids Res 29, 2766-2771.
30. Amack, J. D., Paguio, A. P., and Mahadevan, M. S. (1999) Cis and trans effects of the myotonic dystrophy (DM) mutation in a cell culture model, Hum Mol Genet 8, 1975-1984.
31. Disney, M. D., Labuda, L. P., Paul, D. J., Poplawski, S. G., Pushechnikov, A., Tran, T., Velagapudi, S. P., Wu, M., and Childs-Disney, J. L. (2008) Two-dimensional combinatorial screening identifies specific aminoglycoside-RNA internal loop partners, J Am Chem Soc 130, 11185-11194.
32. Parkesh, R., Childs-Disney, J. L., Nakamori, M., Kumar, A., Wang, E., Wang, T., Hoskins, J., Tran, T., Housman, D. E., Thornton, C. A., and Disney, M. D. (2012) Design of a bioactive small molecule that targets the myotonic dystrophy type 1 RNA via an RNA motif-ligand database & chemical similarity searching, J Am Chem Soc 134-4731-4742.
33. Velagapudi, S. P., Seedhouse, S. J., French, J., and Disney, M. D. (2011) Defining the RNA Internal Loops Preferred by Benzimidazole Derivatives via Two-Dimensional Combinatorial Screening and Computational Analysis, Journal of the American Chemical Society 133, 10111-10118.
34. Velagapudi, S. P., Pushechnikov, A., Labuda, L. P., French, J. M., and Disney, M. D. (2012) Probing a 2-Aminobenzimidazole Library for Binding to RNA Internal Loops via Two-Dimensional Combinatorial Screening, ACS Chemical Biology 7, 1902-1909.
35. Tran, T., and Disney, M. D. (2012) Identifying the preferred RNA motifs and chemotypes that interact by probing millions of combinations, Nat Commun 3, 1125.
36. Cho, J., and Rando, R. R. (2000) Specific binding of Hoechst 33258 to site 1 thymidylate synthase mRNA, Nucleic Acids Res 28, 2158-2163.
37. Pushechnikov, A., Lee, M. M., Childs-Disney, J. L., Sobczak, K., French, J. M., Thornton, C. A., and Disney, M. D. (2009) Rational design of ligands targeting triplet repeating transcripts that cause RNA dominant disease: application to myotonic muscular dystrophy type 1 and spinocerebellar ataxia type 3, J Am Chem Soc 131, 9767-9779.
38. Willett, P. (2011) Similarity searching using 2D structural fingerprints, Methods Mol Biol 672. 133-158.
39. Hawkins, P. C., Skillman, A. G., and Nicholls, A. (2007) Comparison of shape-matching and docking as virtual screening tools, J Med Chem 50, 74-82.
40. Leeson, P. (2012) Drug discovery: Chemical beauty contest, Nature 481, 455-456.
41. Baell, J. B., and Holloway, G. A. (2010) New substructure filters for removal of pan assay interference compounds (PAINS) from screening libraries and for their exclusion in bioassays, J Med Chem 53, 2719-2740.
42. Bruns, R. F., and Watson, I. A. (2012) Rules for identifying potentially reactive or promiscuous compounds, J Med Chem 55, 9763-9772.
43. Paul, S., Dansithong, W., Kim, D., Rossi, J., Webster, N. J., Comai, L., and Reddy, S. (2006) Interaction of muscleblind, CUG-BP1 and hnRNP H proteins in DM1-associated aberrant IR splicing, Embo J 25, 4271-4283.
44. Cardani, R., Mancinelli, E., Rotondo, G., Sansone, V., and Meola, G. (2006) Muscleblind-like protein 1 nuclear sequestration is a molecular pathology marker of DM1 and DM2, Eur J Histochem 50, 177-182.
45. Jiang, H., Mankodi, A., Swanson, M. S., Moxley, R. T., and Thornton, C. A. (2004) Myotonic dystrophy type 1 is associated with nuclear foci of mutant RNA, sequestration of muscleblind proteins and deregulated alternative splicing in neurons, Hum Mol Genet 13, 3079-3088.
46. Chen, C. Z., Sobczak, K., Hoskins, J., Southall, N., Marugan, J. J., Zheng, W., Thornton, C. A., and Austin, C. P. (2012) Two high-throughput screening assays for aberrant RNA-protein interactions in myotonic dystrophy type 1, Anal Bioanal Chem 402, 1889-1898.
47. Faustino, N. A., and Cooper, T. A. (2003) Pre-mRNA splicing and human disease, Genes Dev 17, 419-437.
48. Yang, W.-Y., Wilson, H. D., Velagapudi, S. P., and Disney, M. D. (2015) Inhibition of Non-ATG Translational Events in Cells via Covalent Small Molecules Targeting RNA, Journal of the American Chemical Society 137, 5336-5345.
49. Guan, L., and Disney, M. D. (2013) Covalent small-molecule-RNA complex formation enables cellular profiling of small-molecule-RNA interactions, Angewande Chemie (International ed. in English) 52, 10.1002/anie.201301639.
50. Childs-Disney, J. L., Stepniak-Konieczna, E., Tran, T., Yildirim, I., Park, H., Chen, C. Z., Hoskins, J., Southall, N., Marugan, J. J., Patnaik, S., Zheng, W., Austin, C. P., Schatz, G. C., Sobczak, K., Thornton, C. A., and Disney, M. D. (2013) Induction and reversal of myotonic dystrophy type 1 pre-mRNA splicing defects by small molecules, Nat Commun 4, 2044.
51. Coonrod, L. A., Nakamori, M., Wang, W., Carrell, S., Hilton, C. L., Bodner, M. J., Siboni, R. B., Docter, A. G., Haley, M. M., Thornton, C. A., and Berglund, J. A. (2013) Reducing Levels of Toxic RNA with Small Molecules, ACS Chemical Biology 8, 2528-2537.
52. Mastroyiannopoulos, N. P., Feldman, M. L., Uney, J. B., Mahadevan, M. S., and Phylactou, L. A. (2005) Woodchuck post-transcriptional element induces nuclear export of myotonic dystrophy 3′ untranslated region transcripts, EMBO Rep 6, 458-463.
53. Amack, J. D., and Mahadevan, M. S. (2001) The myotonic dystrophy expanded CUG repeat tract is necessary but not sufficient to disrupt C2C12 myoblast differentiation, Hum Mol Genet 10, 1879-1887.
54. Childs-Disney, J. L., Hoskins, J., Rzuczek, S. G., Thornton, C. A., and Disney, M. D. (2012) Rationally designed small molecules targeting the RNA that causes myotonic dystrophy type 1 are potently bioactive, ACS Chem Biol 7, 856-862.
55. Mankodi, A., Teng-Umnuay, P., Kr m, M., Henderson, D., Swanson, M., and Thornton, C. A. (2003) Ribonuclear inclusions in skeletal muscle in myotonic dystrophy types 1 and 2, Ann Neurol 54, 760-768.
56. Ketley, A., Chen, C. Z., Li, X., Arya, S., Robinson, T. E., Granados-Riveron, J., Udosen, I., Morris, G. E., Holt, I., Furling, D., Chaouch, S., Haworth, B., Southall, N., Shinn, P., Zheng, W., Austin, C. P., Hayes, C. J., and Brook, J. D. (2014) High-content screening identifies small molecules that remove nuclear foci, affect MBNL distribution and CELF1 protein levels via a PKC-independent pathway in myotonic dystrophy cell lines, Human Molecular Genetics 23, 1551-1562.
57. O'Leary, D. A., Vargas, L., Sharif, O., Garcia, M. E., Sigal, Y. J., Chow, S.-K., Schmedt, C., Caldwell, J. S., Brinker, A., and Engels, I. H. (2010) HTS-Compatible Patient-Derived Cell-Based Assay to Identify Small Molecule Modulators of Aberrant Splicing in Myotonic Dystrophy Type 1, Current Chemical Genomics 4, 9-18.
58. Blount, K., Puskarz, J., Penchovsky, R., and Breaker, R. (2006) Development and Application of a High-Throughput Assay for glmS Riboswitch Activators, RNA Biology 3, 77-81.
59. Bandyopadhyay, S., Ni, J., Ruggiero, A., Walshe, K., Rogers, M. S., Chattopadhyay, N., Glicksman, M. A., and Rogers, J. T. (2006) A High-Throughput Drug Screen Targeted to the 5′Untranslated Region of Alzheimer Amyloid Precursor Protein mRNA, Journal of Biomolecular Screening 11, 469-480.
60. Ross, N. M., S R; Le, H; Burbank, J; Cahill, C; Germain, A; MacPherson, L; Bittker, J; Palmer, M; Rogers, J; Schreiber, S L. (2011) Identification of a small molecule that selectively inhibits alpha-synuclein translational expression, National Center for Biotechnology Information (US), Bethesda, Md.
61. Garcia-Lopez, A., Monferrer, L., Garcia-Alcover, I., Vicente-Crespo, M., Alvarez-Abril, M. C., and Artero, R. D. (2008) Genetic and Chemical Modifiers of a CUG Toxicity Model in Drosophila, PLoS ONE 3, e1595.
62. Clark, A. M., and Labute, P. (2008) Detection and Assignment of Common Scaffolds in Project Databases of Lead Molecules, Journal of Medicinal Chemistry 52, 469-483.
63. Warf, M. B., Nakamori, M., Matthys, C. M., Thornton, C. A., and Berglund, J. A. (2009) Pentamidine reverses the splicing defects associated with myotonic dystrophy, Proc Natl Acad Sci USA 106, 18551-18556.
64. Liu, J., Zhang, Q., Xia, Q., Dong, J., and Xu, Q. (2012) Synthesis, characterization and properties of polyimides derived from a symmetrical diamine containing his-benzimidazole rings, Polymer Degradation and Stability 97, 987-994.
65. Holt, I., Mittal, S., Furling, D., Butler-Browne, G. S., David Brook, J., and Morris, G. E. (2007) Defective mRNA in myotonic dystrophy accumulates at the periphery of nuclear splicing speckles, Genes to Cells 12, 1035-1048.
66. Childs-Disney, J. L., Hoskins, J., Rzuczek, S. G., Thornton, C. A., and Disney, M. D. (2012) Rationally Designed Small Molecules Targeting the RNA That Causes Myotonic Dystrophy Type 1 Are Potently Bioactive, ACS Chemical Biology.
67. Mankodi, A, Logigian, E, Callahan, L., McClain, C., White, R., Henderson, D., Krym, M., and Thornton, C. A. (2000) Myotonic dystrophy in transgenic mice expressing an expanded CUG repeat. Science 289, 1769-1773.

We claim:

1. A method for treating myotonic dystrophy type 1 in a human patient suffering therefrom, comprising administering to the patient a therapeutically effective amount of a compound according to formula (I) or a pharmaceutically acceptable salt thereof: 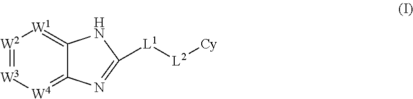
wherein
W¹, W², W³, and W⁴ are independently selected from CR¹ and N, wherein no more than one of W¹, W², W³, and W⁴ is N;
each instance of R¹ is independently selected from the group consisting of H, —NR′R″, —OR′, halo, —C(O)OR′, —C(O)R′, NR′C(O)R″, —NH—C(NH)NH₂, —N═CR′((C₆-C₁₀)arylene)R″;
L¹ and L² are independently selected from the group consisting of a bond, (C₁-C₈)alkylene, —NHC(O)—, (C₆-C₁₀)arylene, heteroarylene;
wherein each (C₁-C₈)alkylene, (C₆-C₁₀)arylene, and heteroarylene are independently and optionally substituted with one or more substituents selected from —OR′, halo, and —NR′R″;
Cy is selected from the group consisting of (C₆-C₁₀)aryl and heteroaryl;
wherein Cy is optionally substituted with one or more substituents selected from the group consisting of —NR′R″, —OR′, halo, —C(O)OR′, —C(O)R′, —NR′C(O)R″, oxo, —NHC(NH)NH₂; and
R′ and R″ are independently selected from the group consisting of H, (C₁-C₆)alkyl), aryl, heteroaryl.

2. The method according to claim 1, wherein each of L¹ and L² is a bond.

3. The method according to claim 1, wherein Cy is selected from the group consisting of phenyl, pyridyl, benzimidazolyl, benzoxazolyl, furanyl, and pyrazinyl.

4. The method according to claim 3, wherein each of W¹, W², W³, and W⁴ is CR¹.

5. The method according to claim 1, wherein the compound of formula (I) is one selected from the following table:

| | |
|---|---|
| 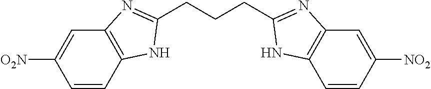 [SMILES: O=[N+]([O-])c1ccc2[nH]c(CCCc3nc4cc([N+](=O)[O-])ccc4[nH]3)nc2c1] | 1 |
| 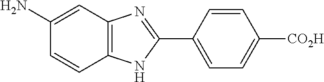 [SMILES: Nc1ccc2[nH]c(-c3ccc(C(=O)O)cc3)nc2c1] | 2 |
| 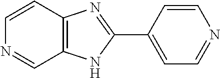 [SMILES: c1cc(-c2nc3ccncc3[nH]2)ccn1] | 3 |
| 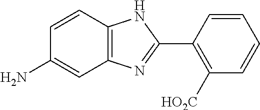 [SMILES: Nc1ccc2[nH]c(-c3ccccc3C(=O)O)nc2c1] | 4 |
| 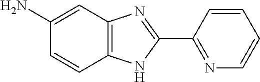 [SMILES: Nc1ccc2[nH]c(-c3ccccn3)nc2c1] | 5 |
| 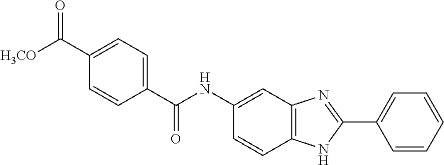 [SMILES: COC(=O)c1ccc(C(=O)Nc2ccc3[nH]c(-c4ccccc4)nc3c2)cc1] | 6 |
| 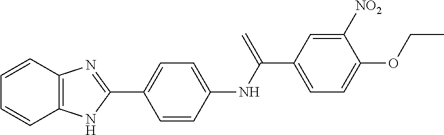 [SMILES: C=C(Nc1ccc(-c2nc3ccccc3[nH]2)cc1)c1ccc(OCC)c([N+](=O)[O-])c1] | 7 |
| 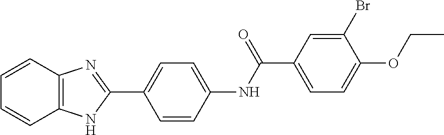 [SMILES: CCOc1ccc(C(=O)Nc2ccc(-c3nc4ccccc4[nH]3)cc2)cc1Br] | 8 |
| 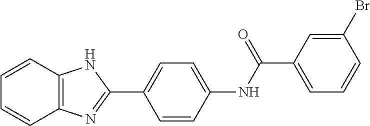 [SMILES: O=C(Nc1ccc(-c2nc3ccccc3[nH]2)cc1)c1cccc(Br)c1] | 9 |
| 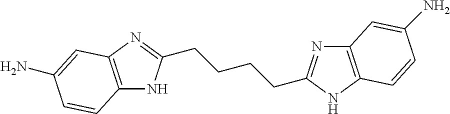 [SMILES: Nc1ccc2[nH]c(CCCCc3nc4cc(N)ccc4[nH]3)nc2c1] | 10 |
| 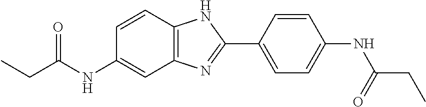 [SMILES: CCC(=O)Nc1ccc(-c2nc3cc(NC(=O)CC)ccc3[nH]2)cc1] | 11 |
| 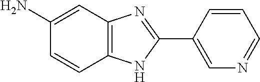 [SMILES: Nc1ccc2[nH]c(-c3cccnc3)nc2c1] | 12 |
| 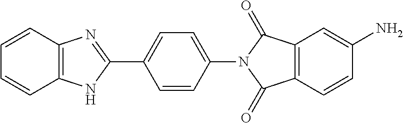 [SMILES: Nc1ccc2c(c1)C(=O)N(c1ccc(-c3nc4ccccc4[nH]3)cc1)C2=O] | 13 |
| 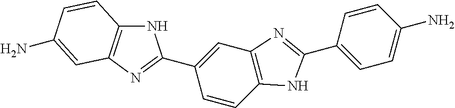 [SMILES: Nc1ccc(-c2nc3cc(-c4nc5cc(N)ccc5[nH]4)ccc3[nH]2)cc1] | 14 |
| 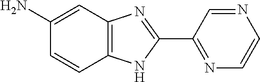 [SMILES: Nc1ccc2[nH]c(-c3cnccn3)nc2c1] | 15 |
| 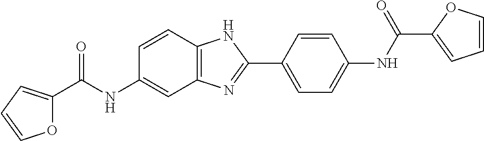 [SMILES: O=C(Nc1ccc(-c2nc3cc(NC(=O)c4ccco4)ccc3[nH]2)cc1)c1ccco1] | 16 |
| 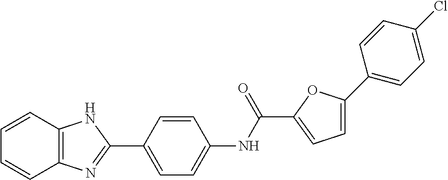 [SMILES: O=C(Nc1ccc(-c2nc3ccccc3[nH]2)cc1)c1ccc(-c2ccc(Cl)cc2)o1] | 17 |
| 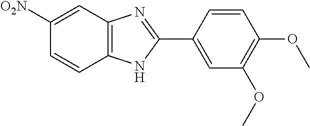 [SMILES: COc1ccc(-c2nc3cc([N+](=O)[O-])ccc3[nH]2)cc1OC] | 18 |
| 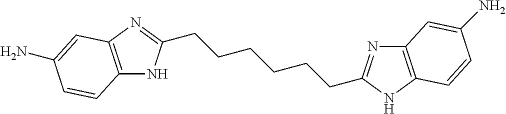 [SMILES: Nc1ccc2[nH]c(CCCCCCc3nc4cc(N)ccc4[nH]3)nc2c1] | 19 |
| 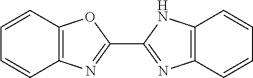 [SMILES: c1ccc2[nH]c(-c3nc4ccccc4o3)nc2c1] | 20 |
| 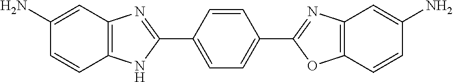 [SMILES: Nc1ccc2[nH]c(-c3ccc(-c4nc5cc(N)ccc5o4)cc3)nc2c1] | 21 |
| 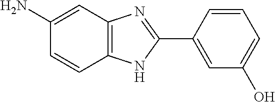 [SMILES: Nc1ccc2[nH]c(-c3cccc(O)c3)nc2c1] | 22 |
| 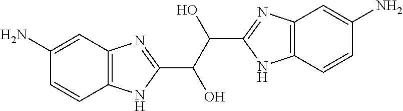 [SMILES: Nc1ccc2[nH]c(C(O)C(O)c3nc4cc(N)ccc4[nH]3)nc2c1] | 23 |
| 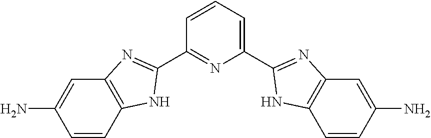 [SMILES: Nc1ccc2[nH]c(-c3cccc(-c4nc5cc(N)ccc5[nH]4)n3)nc2c1] | 24 |
| 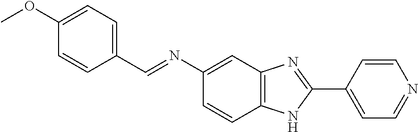 [SMILES: COc1ccc(/C=N/c2ccc3[nH]c(-c4ccncc4)nc3c2)cc1] | 25 |
| 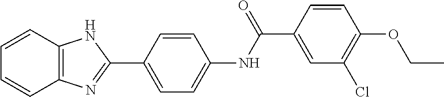 [SMILES: CCOc1ccc(C(=O)Nc2ccc(-c3nc4ccccc4[nH]3)cc2)cc1Cl] | 26 |
| 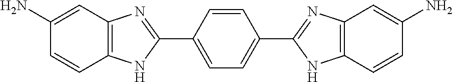 [SMILES: Nc1ccc2[nH]c(-c3ccc(-c4nc5cc(N)ccc5[nH]4)cc3)nc2c1] | 27 |
| 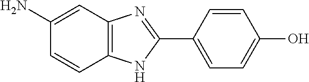 [SMILES: Nc1ccc2[nH]c(-c3ccc(O)cc3)nc2c1] | 28 |
| 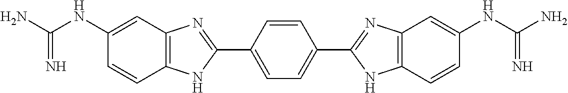 [SMILES: N=C(N)Nc1ccc2[nH]c(-c3ccc(-c4nc5cc(NC(=N)N)ccc5[nH]4)cc3)nc2c1] | 2GN8. |

6. The method according to claim 5, wherein the compound of formula (I) is one selected from the following table:

| | |
|---|---|
| 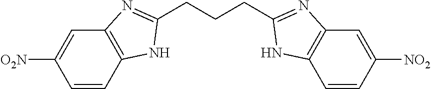 [SMILES: O=[N+]([O-])c1ccc2[nH]c(CCCc3nc4cc([N+](=O)[O-])ccc4[nH]3)nc2c1] | 1 |
| 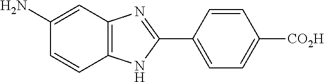 [SMILES: Nc1ccc2[nH]c(-c3ccc(C(=O)O)cc3)nc2c1] | 2 |
| 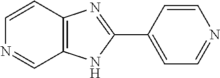 [SMILES: c1cc(-c2nc3ccncc3[nH]2)ccn1] | 3 |
| 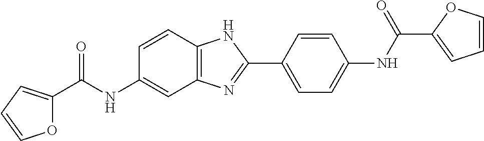 [SMILES: O=C(Nc1ccc(-c2nc3cc(NC(=O)c4ccco4)ccc3[nH]2)cc1)c1ccco1] | 16 |
| 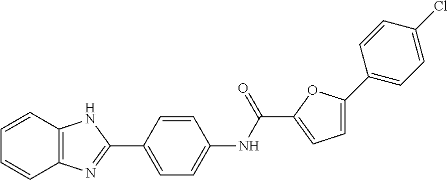 [SMILES: O=C(Nc1ccc(-c2nc3ccccc3[nH]2)cc1)c1ccc(-c2ccc(Cl)cc2)o1] | 17 |
| 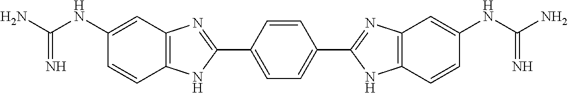 [SMILES: N=C(N)Nc1ccc2[nH]c(-c3ccc(-c4nc5cc(NC(=N)N)ccc5[nH]4)cc3)nc2c1] | 2GN8. |

7. A method for disrupting an RNA-protein complex in a cell, comprising contacting the cell with an effective amount of a compound according to formula (I) or a pharmaceutically acceptable salt thereof: 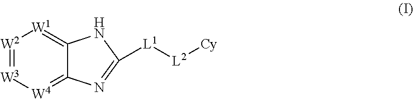
wherein
W¹, W², W³, and W⁴ are independently selected from CR¹ and N, wherein no more than one of W¹, W², W³, and W⁴ is N;
each instance of R¹ is independently selected from the group consisting of H, —NR′R″, —OR′, halo, —C(O)OR′, —C(O)R′, NR′C(O)R″, —NHC(NH)NH₂, —N═CR′ ((C₆₋-C₁₀)arylene)R″;
L¹ and L² are independently selected from the group consisting of a bond, (C₁-C₈)alkylene, —NHC(O)—, (C₆-C₁₀)arylene, heteroarylene;
wherein each (C₁-C₈)alkylene, (C₆-C₁₀)arylene, and heteroarylene are independently and optionally substituted with one or more substituents selected from —OR′, halo, and —NR′R″;
Cy is selected from the group consisting of (C₆-C₁₀)aryl and heteroaryl;
wherein Cy is optionally substituted with one or more substituents selected from the group consisting of —NR′R″, —OR′, halo, —C(O)OR′, —C(O)R′, —NR′C(O)R″, oxo, —NHC(NH)NH₂; and
R′ and R″ are independently selected from the group consisting of H, (C₁-C₆)alkyl), aryl, heteroaryl;
whereby at least a portion of the protein dissociates from the RNA.

8. The method according to claim 7, wherein the RNA comprises a hairpin loop.

9. The method according to claim 8, wherein the RNA comprises an r(CUG)^{exp} segment.

10. The method according to claim 9, wherein the r(CUG)^{exp} segment is r(CUG)₁₂.

11. The method according to claim 7, wherein the RNA is the segment r(CUG)₁₂ and the protein is muscleblind-like 1 protein (MBL1).

12. The method according to claim 7, wherein the cell is a mammalian cell.

13. The method according to claim 12, wherein the mammalian cell is a human cell.

14. A compound according to formula 2GN8: 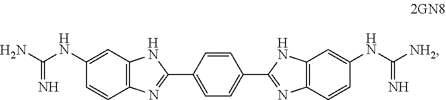 [SMILES: N=C(N)Nc1ccc2nc(-c3ccc(-c4nc5ccc(NC(=N)N)cc5[nH]4)cc3)[nH]c2c1]
or a pharmaceutically acceptable salt thereof.
